SEQUENCE LISTING (36 sequences)

SEQ ID NO: 1 - protein, 118 aa, Artificial
  Humanized sequence
QVQLVQSGAEVKQPGASVKVSCKASGYTFTGYNMYWVRQAPGQGLEWMGYIDPYNGDTTYNQKFKGRVTMTTDTSTSTAYMELRSLRSDDTAVYYCARTSYYGGMDYWGRGTLVTVAS

SEQ ID NO: 2 - DNA, 354 bp, Artificial
  Humanized sequence
caggtgcagctggtgcagagcggagccgaggtgaagcagccaggcgccagcgtgaaggtgtcctgcaaggccagcggctacaccttcaccggctacaacatgtactgggtgcggcaggccccaggccagggcctggaatggatgggctacatcgacccctacaacggcgacaccacctacaaccagaagttcaagggcagggtcaccatgaccaccgacaccagcaccagcaccgcctacatggaactgagaagcctgagatccgacgacaccgccgtgtactactgcgccaggaccagctactacggcggcatggactactggggcaggggcaccctggtgaccgtggcctct

SEQ ID NO: 3 - protein, 118 aa, Artificial
  Humanized sequence
EVQLVQSGAEVKKPGESLKISCKGSGYSFTGYNMYWVRQMPGKGLEWMGYIDPYNGDTTYNQKFKGQVTISADKSISTAYLQWSSLKASDTAMYYCARTSYYGGMDYWGQGTTVTVSS

SEQ ID NO: 4 - protein, 118 aa, Artificial
  Humanized sequence
QVQLVQSGSELKKPGASVKVSCKASGYTFTGYNMYWVRQAPGQGLEWMGYIDPYNGDTTYNQKFKGRFVFSLDTSVSTAYLQISSLKAEDTAVYYCARTSYYGGMDYWGQGTTVTVSS

SEQ ID NO: 5 - protein, 113 aa, Artificial
  Humanized sequence
DIVMTQTPLSSPVTLGQPASISCRSSQSLVHSNGNTYLHWLQQRPGQPPRLLIYKVSNRFSGVPDRFSGSGAGTDFTLKISRVEAEDVGVYYCSQSTHVPRTFGQGTKLEIKR

SEQ ID NO: 6 - DNA, 339 bp, Artificial
  Humanized sequence
gacatcgtgatgacccagacccccctgagcagccccgtgaccctgggccagcccgccagcatcagctgcaggtccagccagagcctggtgcacagcaacggcaacacctacctgcactggctgcagcagagacccggccagccccccaggctgctgatctacaaggtgtccaacaggttcagcggcgtgcccgacaggttctccggcagcggagccggcaccgacttcaccctgaagatcagcagggtggaggccgaggacgtgggcgtgtactactgcagccagagcacccacgtgcccaggaccttcggccagggcaccaagctggaaatcaagagg

SEQ ID NO: 7 - protein, 118 aa, Artificial
  Humanized sequence
QVQLVQSGAEVKQPGASVKVSCKASGYTFTGYNMYWVRQAPGQGLEWMGYIDPYNGDTTYNQKFKGRVTMTTDTSTSTAYMKLRSLRSDDTAVYYCARTSYYGGMDYWGRGTLVTVAS

SEQ ID NO: 8 - DNA, 354 bp, Artificial
  Humanized sequence
caggtgcagctggtgcagagcggagccgaggtgaagcagccaggcgccagcgtgaaggtgtcctgcaaggccagcggctacaccttcaccggctacaacatgtactgggtgcggcaggccccaggccagggcctggaatggatgggctacatcgacccctacaacggcgacaccacctacaaccagaagttcaagggcagggtcaccatgaccaccgacaccagcaccagcaccgcctacatgaaactgagaagcctgagatccgacgacaccgccgtgtactactgcgccaggaccagctactacggcggcatggactactggggcaggggcaccctggtgaccgtggcctct

SEQ ID NO: 9 - protein, 118 aa, Artificial
  Humanized sequence
QVQLVQSGAKVKQPGASVKASCKASGYTFTGYNMYWVRQAPGQGLEWMGYIDPYNGDTTYNQKFKGRVTMTTDTSTSTAYMELRSLRSDDTAVYYCARTSYYGGMDYWGRGTLVTVAS

SEQ ID NO: 10 - DNA, 354 bp, Artificial
  Humanized sequence
caggtgcagctggtgcagagcggagccaaggtgaagcagccaggcgccagcgtgaaggcgtcctgcaaggccagcggctacaccttcaccggctacaacatgtactgggtgcggcaggccccaggccagggcctggaatggatgggctacatcgacccctacaacggcgacaccacctacaaccagaagttcaagggcagggtcaccatgaccaccgacaccagcaccagcaccgcctacatggaactgagaagcctgagatccgacgacaccgccgtgtactactgcgccaggaccagctactacggcggcatggactactggggcaggggcaccctggtgaccgtggcctct

SEQ ID NO: 11 - protein, 118 aa, Artificial
  Humanized sequence
QVQLVQSGAEVKQPGASVKVSCKASGYTFTGYNMYWVRQAPGQGLEWMGYIDPYNGDTTYNQKFKGRATMTTDTSTSTAYMKLRSLRSDDTAVYYCARTSYYGGMDYWGRGTLVTVAS

SEQ ID NO: 12 - DNA, 354 bp, Artificial
  Humanized sequence
caggtgcagctggtgcagagcggagccgaggtgaagcagccaggcgccagcgtgaaggtgtcctgcaaggccagcggctacaccttcaccggctacaacatgtactgggtgcggcaggccccaggccagggcctggaatggatgggctacatcgacccctacaacggcgacaccacctacaaccagaagttcaagggcagggccaccatgaccaccgacaccagcaccagcaccgcctacatgaaactgagaagcctgagatccgacgacaccgccgtgtactactgcgccaggaccagctactacggcggcatggactactggggcaggggcaccctggtgaccgtggcctct

SEQ ID NO: 13 - protein, 118 aa, Artificial
  Humanized sequence
QVQLVQSGAKVKQPGASVKASCKASGYTFTGYNMYWVRQAPGQGLEWMGYIDPYSGDTTYNQKFKGRVTMTTDTSTSTAYMELRSLRSDDTAVYYCARTSYYGGMDYWGRGTLVTVAS

SEQ ID NO: 14 - DNA, 354 bp, Artificial
  Humanized sequence
caggtgcagctggtgcagagcggagccaaggtgaagcagccaggcgccagcgtgaaggcgtcctgcaaggccagcggctacaccttcaccggctacaacatgtactgggtgcggcaggccccaggccagggcctggaatggatgggctacatcgacccctacagcggcgacaccacctacaaccagaagttcaagggcagggtcaccatgaccaccgacaccagcaccagcaccgcctacatggaactgagaagcctgagatccgacgacaccgccgtgtactactgcgccaggaccagctactacggcggcatggactactggggcaggggcaccctggtgaccgtggcctct

SEQ ID NO: 15 - protein, 118 aa, Artificial
  Humanized sequence
QVQLVQSGAEVKQPGASVKVSCKARGYTFTGYNMYWVRQAPGQGLEWMGYIDPYNGDTTYNQKFKGRVTMTTDTSTGTAYMKLRSLRSDDTAVYYCARTSYYGGMDYWGRGTLVTVAS

SEQ ID NO: 16 - DNA, 354 bp, Artificial
  Humanized sequence
caggtgcagctggtgcagagcggagccgaggtgaagcagccaggcgccagcgtgaaggtgtcctgcaaggcccgcggctacaccttcaccggctacaacatgtactgggtgcggcaggccccaggccagggcctggaatggatgggctacatcgacccctacaacggcgacaccacctacaaccagaagttcaagggcagggtcaccatgaccaccgacaccagcaccggcaccgcctacatgaaactgagaagcctgagatccgacgacaccgccgtgtactactgcgccaggaccagctactacggcggcatggactactggggcaggggcaccctggtgaccgtggcctct

SEQ ID NO: 17 - protein, 113 aa, Artificial
  Humanized sequence
DIVMTQTPLSGPVTLGQPASISCRSSQSLVHSNGNTYLHWLQQRPGQPPRLLIYKVSNRFSGVPDRFSGSGAGTDFTLKISRVEAEDVGVYYCSQSTHVPRTFGRGTKLEIKR

SEQ ID NO: 18 - DNA, 339 bp, Artificial
  Humanized sequence
gacatcgtgatgacccagacccccctgagcggccccgtgaccctgggccagcccgccagcatcagctgcaggtccagccagagcctggtgcacagcaacggcaacacctacctgcactggctgcagcagagacccggccagccccccaggctgctgatctacaaggtgtccaacaggttcagcggcgtgcccgacaggttctccggcagcggagccggcaccgacttcaccctgaagatcagcagggtggaggccgaggacgtgggcgtgtactactgcagccagagcacccacgtgcccaggaccttcggccggggcaccaagctggaaatcaagagg

SEQ ID NO: 19 - protein, 113 aa, Artificial
  Humanized sequence
DIVMTQTPLSSPVTLGQPASISCRSSQSLVHGNGNTYLHWLQQRPGQPPRLLIYKVSNRFSGVPDRFSGSGAGTDFTLKISRVEAEDVGVYYCSQSTHVPRTFGQGTKLEIKR

SEQ ID NO: 20 - DNA, 339 bp, Artificial
  Humanized sequence
gacatcgtgatgacccagacccccctgagcagccccgtgaccctgggccagcccgccagcatcagctgcaggtccagccagagcctggtgcacggcaacggcaacacctacctgcactggctgcagcagagacccggccagccccccaggctgctgatctacaaggtgtccaacaggttcagcggcgtgcccgacaggttctccggcagcggagccggcaccgacttcaccctgaagatcagcagggtggaggccgaggacgtgggcgtgtactactgcagccagagcacccacgtgcccaggaccttcggccagggcaccaagctggaaatcaagagg

SEQ ID NO: 21 - protein, 113 aa, Artificial
  Humanized sequence
DIVMTQTPLSSPVTLGQPASISCRSSQSLVHSNGNTYLHWLQQRPGQPPRLLIYKVSNRFSGVPGRFSGSGAGTDFTLKISRVEAEDVGVYYCSQSTHVPRTFGQGTKLEIKR

SEQ ID NO: 22 - DNA, 339 bp, Artificial
  Humanized sequence
gacatcgtgatgacccagacccccctgagcagccccgtgaccctgggccagcccgccagcatcagctgcaggtccagccagagcctggtgcacagcaacggcaacacctacctgcactggctgcagcagagacccggccagccccccaggctgctgatctacaaggtgtccaacaggttcagcggcgtgcccggcaggttctccggcagcggagccggcaccgacttcaccctgaagatcagcagggtggaggccgaggacgtgggcgtgtactactgcagccagagcacccacgtgcccaggaccttcggccagggcaccaagctggaaatcaagagg

SEQ ID NO: 23 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 246 aa, Artificial
  Humanized sequence
QVQLVQSGAEVKQPGASVKVSCKASGYTFTGYNMYWVRQAPGQGLEWMGYIDPYNGDTTYNQKFKGRVTMTTDTSTSTAYMELRSLRSDDTAVYYCARTSYYGGMDYWGRGTLVTVASGGGASGGGGSGGGGSDIVMTQTPLSSPVTLGQPASISCRSSQSLVHSNGNTYLHWLQQRPGQPPRLLIYKVSNRFSGVPDRFSGSGAGTDFTLKISRVEAEDVGVYYCSQSTHVPRTFGQGTKLEIKR

SEQ ID NO: 26 - protein, 5 aa, Artificial
  Humanized sequence
GYNMY

SEQ ID NO: 27 - protein, 17 aa, Artificial
  Humanized sequence
YIDPYNGDTTYNQKFKG

SEQ ID NO: 28 - protein, 9 aa, Artificial
  Humanized sequence
TSYYGGMDY

SEQ ID NO: 29 - protein, 17 aa, Artificial
  Humanized sequence
YIDPYSGDTTYNQKFKG

SEQ ID NO: 30 - DNA, 2128 bp, Homo sapiens
caagacttctctgcattttctgccaaaatctgtgtcagatttaagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 31 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 32 - DNA, 37 bp, Artificial Sequence
  Polynucleotide primer
tagaaagcttaatctagagccaccatgcagcggggcg

SEQ ID NO: 33 - DNA, 24 bp, Artificial Sequence
  Polynucleotide primer
tagaggatccctccatgctgccct

SEQ ID NO: 34 - protein, 16 aa, Artificial
  Humanized sequence
RSSQSLVHSNGNTYLH

SEQ ID NO: 35 - protein, 7 aa, Artificial
  Humanized sequence
KVSNRFS

SEQ ID NO: 36 - protein, 9 aa, Artificial
  Humanized sequence
SQSTHVPRT

(19) United States
(12) Patent Application Publication
(10) Pub. No.: US 2012/0093811 A1
(43) Pub. Date: Apr. 19, 2012

(54) ANTI-VEGF-D ANTIBODIES

(76) Inventors: Jason W. Simmonds, Doreen (AU); Andrew J. Pow, Pasco Vale (AU); Vincent Emil W. Batori, St Kilda West (AU); Irene Koukoulas, Camberwell (AU); George Kopsidas, Preston (AU)

(21) Appl. No.: 13/262,487

(22) PCT Filed: Apr. 1, 2010
(86) PCT No.: PCT/AU10/00376

§ 371 Date: Dec. 27, 2011

Related U.S. Application Data
(60) Provisional application No. 61/166,555, filed on Apr. 3, 2009.

(51) Int. Cl. A61K 39/395; C12N 15/13; C12N 1/21; A61P 17/06; C12N 1/19; C12N 5/10; A61P 35/00; A61P 29/00; C07K 16/22; C12N 1/15

(52) U.S. Cl. 424/133.1; 530/387.3; 536/23.53; 435/252.33; 435/252.35; 435/252.31; 435/252.3; 435/254.3; 435/254.11; 435/254.2; 435/328; 435/419

(57) ABSTRACT

The invention relates to an isolated antibody that specifically binds vascular endothelial growth factor-D (VEGF-D) and to a humanized antibody that specifically binds VEGF-D.

### FIELD

The invention relates to anti-VEGF-D antibodies and uses thereof.

### BACKGROUND

The most recently discovered mammalian member of the vascular endothelial growth factor (VEGF) family, VEGF-D, activates the cell surface receptor tyrosine kinases VEGFR-2 and VEGFR-3. VEGFR-2 and VEGFR-3 are expressed on the endothelial cells of blood vessels and lymphatic vessels during embryogenesis. Thereafter, VEGFR-2 is predominantly expressed on blood vessels, whereas VEGFR-3 is predominantly expressed on lymphatic vessels. Both VEGFR-2 and VEGFR-3 appear to have significant roles in tumor development and spread and in numerous other disease states.

VEGFR-2 is thought to be a critical molecule for signaling angiogenesis, whereas VEGFR-3 appears to signal primarily for lymphangiogenesis. VEGFR-3 can also be up-regulated on growing blood vessels and participate in angiogenic signaling.

VEGF-D is synthesized as a prepro-polypeptide and is activated by proteolytic processing by proprotein convertases. In humans, it is the mature proteolytically processed form of VEGF-D that binds predominantly to VEGFR-2 and VEGFR-3.

Angiogenesis is a fundamental process required for normal growth and development of tissues, and involves the proliferation of new capillaries from pre-existing blood vessels. Angiogenesis is not only involved in embryonic development and normal tissue growth, repair, and regeneration, but is also involved in the female reproductive cycle, establishment and maintenance of pregnancy, and in repair of wounds and fractures.

In addition to angiogenesis which takes place in the normal individual, angiogenic events are involved in a number of pathological processes, notably tumor growth and metastasis, and other conditions in which blood vessel proliferation, especially of the microvascular system, is increased, such as diabetic retinopathy, psoriasis and arthropathies.

Recent studies have demonstrated the critical role of angiogenesis in tumor development and the formation of metastatic tumor deposits. VEGF-D promoted tumor angiogenesis and lymphangiogenesis in a mouse model of cancer, facilitating growth of the primary tumor and spread of tumor cells via the lymphatic vessels to lymph nodes. VEGF-D is expressed in a range of prevalent human cancers and has been reported to be a prognostic indicator of lymphatic involvement and poor patient outcome in some tumor types. Accordingly, the inhibition of tumor angiogenesis and VEGF-D has emerged as a promising new anti-cancer therapeutic modality.

A need exists for an effective, alternative means to prevent, restrict or treat a condition responsive to neutralizing VEGF-D, particularly angiogenesis, and cancer associated therewith.

### SUMMARY

A first aspect provides an isolated antibody that specifically binds vascular endothelial growth factor-D (VEGF-D) comprising a heavy chain variable (V_{H}) domain amino acid sequence provided as SEQ ID NO: 1 or a variant thereof which contains from 1 to 10 amino acid substitutions.

Complementarity determining regions (CDRs) of a humanized heavy chain variable (V_{H}) domain involved in the specific binding to VEGF-D are located at residues 31 to 35 (CDR1), 50 to 66 (CDR2) and 99 to 107 (CDR3) of SEQ ID NO: 1, which correspond to residues 31 to 35, 50 to 66, and 99 to 107, respectively, of SEQ ID NO: 25. Framework regions (FRs) are located at residues 1 to 30 (FR1), 36 to 49 (FR2), 67 to 98 (FR3), and 108 to 118 (FR4) of SEQ ID NO: 1, which correspond to residues 1 to 30, 36 to 49, 67 to 98, and 108 to 118, respectively, of SEQ ID NO: 25.

In some embodiments, it is contemplated that the isolated antibody that specifically binds VEGF-D comprising a V_{H} domain amino acid sequence of SEQ ID NO: 1 contains 1, 2, 3, 4, 5, 6, 7, 8, 9 or 10 amino acid substitutions in one or more of the FR described above. In some embodiments, the V_{H} domain of SEQ ID NO: 1 contains 1, 2, 3, 4, 5 or 6 amino acid substitutions in one or more V_{H} CDR described above.

A second aspect provides a humanized antibody that specifically binds VEGF-D comprising a CDR1 having the amino acid sequence provided as SEQ ID NO: 26, CDR2 having the amino acid sequence provided as SEQ ID NO: 29 and CDR3 having the amino acid sequence provided as SEQ ID NO: 28, wherein CDR1, CDR2 and CDR3 are inserted within a human framework sequence.

In embodiments where the V_{H} domain amino acid sequence is a variant of SEQ ID NO: 1, the variant preferably contains no more than 9, more preferably no more than 8, yet more preferably no more than 7 and yet more preferably no more than 6 amino acid substitutions (relative to SEQ ID NO: 1). For example, the V_{H} domain amino acid sequence is a variant of SEQ ID NO: 1 that may contain no more than 5 or no more than 4 amino acid substitutions. In a preferred embodiment, the V_{H} domain amino acid sequence is a variant of SEQ ID NO: 1 that contains 1, 2 or 3 amino acid substitutions.

In one embodiment, the V_{H} domain amino acid sequence is a variant of SEQ ID NO: 1 that comprises an amino acid substitution at position 1, 2, 3, 5, 9, 10, 11, 12, 13, 16, 17, 18, 19, 20, 23, 24, 25, 28, 40, 43, 46, 48, 55, 63, 65, 67, 68, 69, 70, 71, 72, 73, 74, 75, 76, 77, 81, 82, 83, 84, 87, 88, 89, 93, 95, 99, 100, 110, 112, 113, 114 or 117 (said positions referring to positions in SEQ ID NO: 1). Preferably, the V_{H} domain amino acid sequence is a variant of SEQ ID NO: 1 that comprises an amino acid substitution at position 2, 3, 5, 10, 12, 17, 18, 19, 20, 23, 25, 40, 46, 48, 55, 63, 65, 68, 69, 73, 75, 77, 81, 82, 89, 95, 99, 100, 112, 114, or 117. More preferably, the V_{H} domain amino acid sequence is a variant of SEQ ID NO: 1 that comprises an amino acid substitution at position 10, 20, 25, 55, 77 or 82.

In another embodiment, the V_{H} domain amino acid sequence is a variant of SEQ ID NO: 1 that comprises one or more amino acid substitutions selected from the group consisting of Q1E; V2A; Q3R; V5A; A9S; E10K; V11L; K12R; Q13K; A16E; S17N; S17G; V18M; V18L; K19R; K19T; V20A; V20I; K23E; A24G; S25G; S25R; T28S; A40M; A40V; Q43K; E46G; M48V; N55S; K63R; K65R; R67Q; V68A; T69A; V68F; T69V; M70F; M70I; T71S; T72A; T72L; D73G; T74K; S75N; T76V; T76I; S77G; M81V; M81L; E82K; E82Q; L83I; L83W; R84S; R87K; S88A; D89G; D89E; D89S; V93M; Y95H; T99A; S100G; R110Q; T112A; L113T; V114A; and A117S. Preferably, the V_{H} domain amino acid sequence is a variant of SEQ ID NO: 1 that comprises one or more amino acid substitutions selected from the group consisting of V2A; Q3R; V5A; E10K; K12R; S17N; S17G; V18M; K19R; K19T; V20A; K23E; S25G; S25R; A40V; E46G; M48V; N55S; K63R; K65R; V68A; D73G; S75N; S77G; M81V; E82K; D89G; Y95H; T99A; S100G; T112A,V114A; and A117S.

In a preferred embodiment, the V_{H} domain amino acid sequence is a variant of SEQ ID NO: 1 that comprises one or more amino acid substitutions selected from the group consisting of E10K, V20A, S25R, N55S, V68A, S77G and E82K. In a particularly preferred embodiment, the V_{H} domain amino acid sequence is a variant of SEQ ID NO: 1 comprising one or more amino acid substitutions selected from the group consisting of E10K, V20A and N55S. In another embodiment, the V_{H} domain amino acid sequence is a variant of SEQ ID NO: 1 that comprises at least the amino acid substitution N55S. In yet another embodiment, the V_{H} domain amino acid sequence is a variant of SEQ ID NO: 1 that comprises the three amino acid substitutions E10K, V20A and N55S. In yet another embodiment, the SEQ ID NO: 1 variant comprises the sequence provided as SEQ ID NO: 13.

Examples of preferred SEQ ID NO: 1 variants include sequences comprising the following substitutions: E82K; E10K and V20A; V68A and E82K; and S25R, S77G and E82K.

Examples of preferred SEQ ID NO: 1 variants include the sequences provided as SEQ ID NO: 7; SEQ ID NO: 9; SEQ ID NO: 11; and SEQ ID NO: 15.

The isolated antibody of the first aspect may, in addition to the V_{H} domain, also contain a light chain variable (V_{L}) domain amino acid sequence provided as SEQ ID NO: 5 or a variant thereof which contains from 1 to 10 amino acid substitutions.

CDRs of a humanized light chain variable (V_{L}) domain involved in the specific binding to VEGF-D are located at residues 24 to 39 (CDR1), 55 to 61 (CDR2), and 94 to 102 (CDR3) of SEQ ID NO: 5, which correspond to residues 157 to 172, 188 to 194, and 227 to 235, respectively, of SEQ ID NO: 25. The FRs are located at residues 1 to 23 (FR1), 40 to 54 (FR2), 62 to 93 (FR3), and 103 to 113 (FR4) of SEQ ID NO: 1, which correspond to residues 134 to 156, 173 to 187, 195 to 226, and 236 to 246, respectively, of SEQ ID NO: 25.

In some embodiments, it is contemplated that the isolated antibody that specifically binds VEGF-D comprising a V_{L} domain amino acid sequence of SEQ ID NO: 5 contains 1, 2, 3, 4, 5, 6, 7, 8, 9 or 10 amino acid substitutions in one or more of the FRs described above. In some embodiments, the V_{H} domain of SEQ ID NO: 1 contains 1, 2, 3, 4, 5 or 6 amino acid substitutions in one or more V_{H} CDRs described above.

In a preferred embodiment, the antibody of the first or second aspect comprises a V_{H} domain and a V_{L} domain. In some variations, the antibody of the first aspect comprises one, two, or three V_{H} CDRs set out in SEQ ID NOs: 26, 29 or 28, and/or comprises one, two, or three V_{L} CDRs set out in SEQ ID NOs: 34, 35 or 36. In some variations, in addition to the CDR set out in SEQ ID NO: 29, the antibody of the second aspect comprises one or two V_{H} CDRs set out in SEQ ID NOs: 26 or 28, and/or comprises one, two, or three V_{L} CDRs set out in SEQ ID NOs: 34, 35 or 36.

In another embodiment, the antibody of the first or second aspect comprises at least one CDR of a light chain variable region of a VEGF-D-specific antibody, wherein the light chain variable region comprises an amino acid sequence at least 90% identical to the CDR sequences set out in SEQ ID NOs: 34, 35 or 36. In some variations, the antibody comprises at least one CDR, wherein the at least one CDR is at least 90%, 95%, 96%, 97%, 98%, or 99% identical to the CDRs set out in SEQ ID NOs: 34, 35 or 36.

In another embodiment, the antibody of the first or second aspect comprises at least one CDR of a heavy chain variable region of a VEGF-D-specific antibody, wherein the heavy chain variable region comprises an amino acid sequence at least 90% identical to the CDR sequences set out in SEQ ID NO: 26, 29, or 28, provided that the CDR sequences of an antibody of the second aspect are not identical to SEQ ID NO: 26, 27 and 28. In some variations, the at least one CDR is at least 90%, 95%, 96%, 97%, 98%, or 99% identical to the polypeptide of SEQ ID NO: 26, 29 or 28, provided that the CDR sequences of an antibody of the second aspect are not identical to SEQ ID NO: 26, 27 and 28.

In embodiments wherein the V_{L} domain amino acid sequence is a variant of SEQ ID NO: 5, the variant preferably contains no more than 9, more preferably no more than 8, yet more preferably no more than 7 and yet more preferably no more than 6 amino acid substitutions. For example, the V_{L} domain amino acid sequence is a variant of SEQ ID NO: 5 that may contain no more than 5, no more than 4, or no more than 3 amino acid substitutions. In a preferred embodiment, the V_{L} domain amino acid sequence is a variant of SEQ ID NO: 5 that contains 1 or 2 amino acid substitutions.

In a further embodiment, the V_{L} domain amino acid sequence is a variant of SEQ ID NO: 5 that comprises an amino acid substitution at position 5, 11, 13, 25, 32, 58, 60, 65, 75, 79, 88, 97, 105 or 108, which correspond with positions 138, 144, 146, 158, 165, 191, 193, 198, 208, 212, 221, 230, 238 or 241, respectively, in SEQ ID NO: 25. In another embodiment, the V_{L} domain amino acid sequence is a variant of SEQ ID NO: 5 that comprises one or more amino acid substitutions selected from the group consisting T5I; S11G; V13A; S25T; S32G; N58S; F60S; D65G; T75A; K79R; V88M; T97A; Q105R; and T108A (that correspond with T138I, S144G, V146A, S158T, S165G, N191S, F193S, D198G, T208A, K212R, V221M, T230A, Q238R and T241A, respectively, in SEQ ID NO: 25).

In a preferred embodiment, the V_{L} domain amino acid sequence is a variant of SEQ ID NO: 5 that comprises one or more amino acid substitutions selected from the group consisting of S11G; S32G; D65G; and Q105R (that correspond with S144G, S165G, D198G and Q238R, respectively, in SEQ ID NO: 25).

Examples of preferred SEQ ID NO: 5 variants include sequences comprising one or more substitutions selected from the group consisting of S11G and Q105R; S32G; and D65G (that correspond with S144G and Q238R; S165G; and D198G, respectively, in SEQ ID NO: 25).

Examples of preferred SEQ ID NO: 5 variants include the sequences provided as SEQ ID NO: 17; SEQ ID NO: 19; or SEQ ID NO: 21.

Examples of preferred combinations of V_{H} and V_{L} domains include:
(a) SEQ ID NO: 1 and SEQ ID NO: 5;
(b) SEQ ID NO: 1 variant E82K (SEQ ID NO: 7) and SEQ ID NO: 5;
(c) SEQ ID NO: 1 variant E10K and V20A (SEQ ID NO: 9) and SEQ ID NO: 5;
(d) SEQ ID NO: 1 variant V68A and E82K (SEQ ID NO: 11) and SEQ ID NO: 5;
(e) SEQ ID NO: 1 variant E10K, V20A and N55S (SEQ ID NO: 13) and SEQ ID NO: 5;
(f) SEQ ID NO: 1 variant S25R, S77G and E82K (SEQ ID NO: 15) and SEQ ID NO: 5;
(g) SEQ ID NO: 1 variant E82K (SEQ ID NO: 7) and SEQ ID NO: 5 variant S11G and Q105R (SEQ ID NO: 17);
(h) SEQ ID NO: 1 variant E82K (SEQ ID NO: 7) and SEQ ID NO: 5 variant S32G (SEQ ID NO: 19);
(i) SEQ ID NO: 1 variant E10K, V20A and N55S (SEQ ID NO: 13) and SEQ ID NO: 5 variant S11G and Q105R (SEQ ID NO: 17); and
(j) SEQ ID NO: 1 variant E10K, V20A and N55S (SEQ ID NO: 13) and SEQ ID NO: 5 variant D65G (SEQ ID NO: 21).

In one embodiment, the dissociation constant (K_{D}) of the antibody of the first or second aspect for mature VEGF-D (as defined below in greater detail) is less than about 40 nM, or less than about 35 nM, or less than about 30 nM, or less than about 25 nM, or less than about 20 nM, or less than about 15 nM, or less than about 10 nM, or less than about 5 nM, or less than about 4 nM, or less than about 3 nM, or less than about 2 nM, or less than about 1 nM, or less than about 900 pM, or less than about 800 pM, or less than about 700 pM, or less than about 600 pM, or less than about 500 pM, or less than about 400 pM, or less than about 300 pM, or less than about 200 pM, or less than about 100 pM, or less than about 50 pM, or less than about 10 pM, or less than about 1 pM.

In a related embodiment, the antibody of the invention retains binding affinity of at least 10⁻⁷, 10⁻⁸, 10⁻⁹ M or higher. In a further embodiment, the invention includes compositions of isolated antibody that binds to VEGF-D with an affinity Kd ranging from about 10⁻⁸ M to 10⁻¹² M, or about 10⁻⁹ M to 10⁻¹² M, or about 10⁻⁹ M to 10⁻¹¹ M; in a related embodiment, the invention contemplates the use of such compositions to treat disorders associated with VEGF-D as described herein. Affinity is measured using techniques well-known in the art, including but not limited to, surface plasmon resonance. In a still further embodiment, the antibody compositions are sterile.

In a preferred embodiment, the antibody of the first or second aspect is monoclonal.

In another embodiment, the antibody of the first or second aspect comprises one or more constant domain(s), such as a heavy chain constant region and/or a light chain constant region.

In some variations, the light chain constant region is a kappa or lambda light chain. In some variations, the heavy chain constant region is selected from the group consisting of a constant region from an IgM chain, an IgG chain, an IgA chain, an IgE chain, an IgD chain, fragments thereof, and combinations thereof In some variations, the heavy chain constant region comprises an IgG chain selected from the group consisting of IgG1, IgG2, IgG3, IgG4, fragments thereof, and combinations thereof. In some variations, the constant region comprises at least one of CH1, CH2, and CH3 regions of a human IgG1 heavy chain constant region.

In one embodiment, the antibody comprises one or more constant domain(s) of IgG isotype. In another embodiment, the antibody comprises a human constant domain of IgG isotype. In a particular embodiment, the antibody comprises a heavy chain constant region amino acid sequence provided as SEQ ID NO: 23. In the case of the antibody also comprising a V_{L} domain, the antibody may also comprise a light chain constant region amino acid sequence provided as SEQ ID NO: 24.

In yet another embodiment, the antibody of the first or second aspect comprises a Fab, Fab′, F(ab′)₂, Fv, scFV, diabody, triabody, tetrabody, miniantibody, minibody, or isolated V_{H} domain. For some variations, including but not limited to SCFV, one or more linker peptides may be used to attach antibody peptide sequences.

The antibody of the first or second aspect comprises an N-terminus and a C-terminus, and in one embodiment, the antibody may be truncated at the N-terminus or the C-terminus, where the truncated antibody still retains VEGF-D binding activity. N- and/or C-Terminal truncations of 1, 2, 3, 4, 5, 6, 7, 8, 9, or 10 amino acids (e.g., relative to SEQ ID NOs: 1 or 5 or other sequences described herein) are specifically contemplated.

According to another embodiment of the first or second aspect, the antibody is chimeric and/or includes or is attached to or conjugated to a heterologous agent. The heterologous agent may be a therapeutic agent or a diagnostic agent, for example, a detectable label. The heterologous agent may be a cytotoxic agent, a radioisotope, an enzyme, a fluorescent label, a luminescent label, a bioluminescent label, a chemiluminescent label, or biotin.

A third aspect provides an isolated nucleic acid molecule that comprises a nucleotide sequence encoding an antibody according to the first or second aspect.

A fourth aspect provides a vector comprising a nucleic acid molecule according to the third aspect. Exemplary vectors include expression vectors suitable for recombinant expression of encoded polypeptides or antibodies in cultured cell expression systems; and expression vectors suitable for in vivo expression in a mammalian organism, such as a human, preferably in therapeutically effective amounts.

A fifth aspect provides an isolated host cell comprising a nucleic acid molecule that comprises a nucleotide sequence encoding an antibody according to the first or second aspect, or a vector according to the fourth aspect. According to one embodiment, the host cell is eukaryotic. In one embodiment, the host cell is mammalian.

A sixth aspect provides a method of producing an antibody comprising the steps of: culturing a host cell comprising a nucleic acid molecule that comprises a nucleotide sequence encoding an antibody according to the first or second aspect or a vector according to the fourth aspect, wherein the cell produces an antibody comprising the encoded antibody and the antibody specifically binds VEGF-D; and obtaining the antibody. In one embodiment, the antibody is secreted from the cell into a culture medium from which the antibody is obtained. In another embodiment, the antibody is obtained from the cell.

A seventh aspect provides an antibody produced according to the method of the sixth aspect.

An eighth aspect provides a pharmaceutical composition comprising an antibody according to the first or second aspect, a nucleic acid according to the third aspect, a vector according to the fourth aspect, or a host cell according to the fifth aspect, and a pharmaceutically acceptable carrier.

A ninth aspect provides a kit comprising an antibody according to the first or second aspect or a nucleic acid molecule of the third aspect and instructions for use.

A tenth aspect provides a method of inhibiting angiogenesis comprising the step of administering to an individual in need thereof a therapeutically effective amount of an antibody according to the first or second aspect, a nucleic acid molecule according to the third aspect, a vector according to the fourth aspect, a host cell according to the fifth aspect, or a pharmaceutical composition according to the eighth aspect. In certain embodiments, the antibody is administered at a dose between about 10 ng/kg to 100 mg/kg, 2 μg/kg to 50 mg/kg, 0.1 mg/kg to 30 mg/kg, or 0.1 mg/kg to 10 mg/kg.

A related variation is a method of inhibiting VEGF-D stimulation of either or both of its receptors, VEGFR-3 and VEGFR-2, in cells that express these receptors.

An eleventh aspect provides a method of diagnosing angiogenesis comprising the steps of contacting a test sample and a reference sample with an antibody according to the first or second aspect; determining specific binding of the antibody to any VEGF-D present in the test sample thereby providing a first VEGF-D expression level; determining specific binding of the antibody to VEGF-D present in the reference sample thereby providing a second VEGF-D expression level; determining the first VEGF-D expression level in the test sample; determining the second VEGF-D expression level in the reference sample; and comparing the first VEGF-D expression level in the test sample to the second VEGF-D expression level in the reference sample, wherein a greater first VEGF-D expression level in the test sample compared with the second VEGF-D expression level in the reference sample is indicative of increased angiogenesis in the test sample.

A related variation of the invention is a method of detecting, quantitatively detecting, or measuring VEGF-D in a sample that comprises contacting the sample with an antibody according to the first or second aspect; and detecting, quantitatively detecting, or measuring VEGF-D in the sample by measuring the antibody bound to the VEGF-D. The measuring may be facilitated by a label on the antibody, or use of a second detecting antibody, for example.

A twelfth aspect provides use of an antibody according to the first or second aspect, a nucleic acid molecule according to the third aspect, a vector according to the fourth aspect, a host cell according to the fifth aspect, or a pharmaceutical composition according to the eighth aspect for inhibiting or diagnosing angiogenesis, or for inhibiting VEGF-D mediated stimulation of VEGFR-2 or R-3.

A thirteenth aspect provides use of an antibody according to the first or second aspect, a nucleic acid molecule according to the third aspect, a vector according to the fourth aspect, or a host cell according to the fifth aspect in the manufacture of a medicament for inhibiting angiogenesis.

A fourteenth aspect provides a method of detecting the presence of VEGF-D in a sample comprising the step of contacting the sample with an antibody according to the first or second aspect and determining specific binding of the antibody to any VEGF-D present in the sample thereby providing a VEGF-D expression level. In one embodiment, the method comprises the step of comparing the VEGF-D expression level in the sample to a VEGF-D expression level in a reference sample, wherein greater VEGF-D expression level in the sample compared with the reference sample is indicative of increased angiogenesis.

In embodiments of the eleventh, twelfth, thirteenth and fourteenth aspects, the angiogenesis is a feature of dysregulated angiogenesis, dysregulated lymphangiogenesis, cancer, rheumatoid arthritis, psoriasis, lymphangiolieomyomatosis, or other inflammatory condition.

A fifteenth aspect provides an isolated antibody that specifically binds VEGF-D comprising:

a light chain variable (V_{L}) domain amino acid sequence provided as SEQ ID NO: 5 or a variant thereof which contains from 1 to 10 amino acid substitutions.

In one embodiment, the antibody of the fifteenth aspect comprises an isolated V_{L} domain.

In a sixteenth aspect, the invention provides an isolated polypeptide comprising one or more of the antibody sequences described above. In one embodiment, the polypeptide comprises all or part of the amino acid sequence set out in SEQ ID NO: 1 or SEQ ID NO: 5 or variants thereof described above. In a related embodiment, the polypeptide comprises one, two, three, four, five or six of the V_{H} and/or V_{L} CDR of a VEGF-D-specific antibody or variants thereof described herein. It is further contemplated that the polypeptide comprises a heavy and/or light chain constant region or fragment thereof.

The person skilled in the art will appreciate that any embodiment described in relation to one aspect of the invention may apply also to the other aspects of the invention.

The original claims appended hereto are hereby incorporated by reference into the summary of the invention. Inventions described as uses should be considered to also constitute a description of methods or processes of using, and vice versa, in view of different jurisdictions preferences for characterizing inventions differently.

The foregoing summary is not intended to define every aspect of the invention, and the heading “Summary” is not intended to be limiting in any way; additional aspects of the invention and further details of the invention are described in other sections, such as the Drawing or Detailed Description. The entire document is intended to be related as a unified disclosure, and it should be understood that all combinations of features described herein are contemplated, even if the combination of features are not found together in the same sentence, or paragraph, or section of this document. To provide an example, where protein/antibody therapy is described, embodiments involving polynucleotide (nucleic acid) therapy (using polynucleotides/nucleic acids that encode the protein/antibody) are always specifically contemplated, and the reverse also is true.

In addition to the foregoing, the invention includes, as an additional aspect, all embodiments of the invention narrower in scope in any way than the variations specifically mentioned above. Embodiments summarized in the Summary section are frequently further described, with preferred variations, in the Detailed Description, and these variations are part of the invention incorporated into the Summary by reference. With respect to aspects of the invention described as a genus, all individual species are individually considered separate aspects of the invention. With respect to aspects of the invention that are described with reference to exemplary numerical values, it should be understood that such values are intended to describe ranges or sub-ranges that include the recited values. With respect to aspects described with numerical ranges, it should be understood that all sub-ranges are contemplated.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE FIGURES

FIG. 1 provides an amino acid sequence (SEQ ID NO: 1) of a humanized heavy chain variable (V_{H}) domain that specifically binds VEGF-D. The CDRs are located at residues 31 to 35 (CDR1), 50 to 66 (CDR2) and 99 to 107 (CDR3) of SEQ ID NO: 1, which correspond to residues 31 to 35, 50 to 66, and 99 to 107, respectively, of SEQ ID NO: 25. The FRs are located at residues 1 to 30 (FR1), 36 to 49 (FR2), 67 to 98 (FR3), and 108 to 118 (FR4) of SEQ ID NO: 1, which correspond to residues 1 to 30, 36 to 49, 67 to 98, and 108 to 118, respectively, of SEQ ID NO: 25.

FIG. 2 provides a nucleic acid sequence (SEQ ID NO: 2) encoding the amino acid sequence depicted in FIG. 1.

FIG. 3 provides an amino acid sequence (SEQ ID NO: 3) of a humanized heavy chain variable (V_{H}) domain that specifically binds VEGF-D. The CDRs are located at residues 31 to 35 (CDR1), 50 to 66 (CDR2) and 99 to 107 (CDR3) of SEQ ID NO: 3.

FIG. 4 provides an amino acid sequence (SEQ ID NO: 4) of a humanized heavy chain variable (V_{H}) domain that specifically binds VEGF-D. The CDRs are located at residues 31 to 35 (CDR1), 50 to 66 (CDR2) and 99 to 107 (CDR3) of SEQ ID NO: 4.

FIG. 5 provides an amino acid sequence (SEQ ID NO: 5) of a humanized light chain variable (V_{L}) domain that specifically binds VEGF-D. The CDRs are located at residues 24 to 39 (CDR1), 55 to 61 (CDR2), and 94 to 102 (CDR3) of SEQ ID NO: 5, which correspond to residues 157 to 172, 188 to 194, and 227 to 235, respectively, of SEQ ID NO: 25. The FRs are located at residues 1 to 23 (FR1), 40 to 54 (FR2), 62 to 93 (FR3), and 103 to 113 (FR4) of SEQ ID NO: 1, which correspond to residues 134 to 156, 173 to 187, 195 to 226, and 236 to 246, respectively, of SEQ ID NO: 25.

FIG. 6 provides a nucleic acid sequence (SEQ ID NO: 6) encoding the amino acid sequence depicted in FIG. 5.

FIG. 7 provides an amino acid sequence of a variant (SEQ ID NO: 7) of the humanized V_{H} domain of FIG. 1 (SEQ ID NO: 1). This V_{H} amino acid sequence is expressed by clones F61, HU75 and HH41.

FIG. 8 provides a nucleic acid sequence (SEQ ID NO: 8) encoding the amino acid sequence depicted in FIG. 7. The nucleic acid sequence is present in clones F61, HU75 and HH41.

FIG. 9 provides an amino acid sequence of a variant (SEQ ID NO: 9) of the humanized V_{H} domain of FIG. 1 (SEQ ID NO: 1). This V_{H} amino acid sequence is expressed by clone A63.

FIG. 10 provides a nucleic acid sequence (SEQ ID NO: 10) encoding the amino acid sequence depicted in FIG. 9. The nucleic acid sequence is present in clone A63.

FIG. 11 provides an amino acid sequence of a variant (SEQ ID NO: 11) of the humanized V_{H} domain of FIG. 1 (SEQ ID NO: 1). This V_{H} amino acid sequence is expressed by clone AW61.

FIG. 12 provides a nucleic acid sequence (SEQ ID NO: 12) encoding the amino acid sequence depicted in FIG. 11. The nucleic acid sequence is present in clone AW61.

FIG. 13 provides an amino acid sequence of a variant (SEQ ID NO: 13) of the humanized V_{H} domain of FIG. 1 (SEQ ID NO: 1). This V_{H} amino acid sequence is expressed by clones DI26, HH69 and HX28.

FIG. 14 provides a nucleic acid sequence (SEQ ID NO: 14) encoding the amino acid sequence depicted in FIG. 13. The nucleic acid sequence is present in clones DI26, HH69 and HX28.

FIG. 15 provides an amino acid sequence of a variant (SEQ ID NO: 15) of the humanized V_{H} domain of FIG. 1 (SEQ ID NO: 1). This V_{H} amino acid sequence is expressed by clone HW78.

FIG. 16 provides a nucleic acid sequence (SEQ ID NO: 16) encoding the amino acid sequence depicted in FIG. 15. The nucleic acid sequence is present in clone HW78.

FIG. 17 provides an amino acid sequence of a variant (SEQ ID NO: 17) of the humanized V_{L} domain of FIG. 5 (SEQ ID NO: 5). This V_{L} amino acid sequence is expressed by clones HU75 and HH69.

FIG. 18 provides a nucleic acid sequence (SEQ ID NO: 18) encoding the amino acid sequence depicted in FIG. 17. The nucleic acid sequence is present in clones HU75 and HH69.

FIG. 19 provides an amino acid sequence of a variant (SEQ ID NO: 19) of the humanized V_{L} domain of FIG. 5 (SEQ ID NO: 5). This V_{L} amino acid sequence is expressed by clone HH41.

FIG. 20 provides a nucleic acid sequence (SEQ ID NO: 20) encoding the amino acid sequence depicted in FIG. 19. The nucleic acid sequence is present in clone HH41.

FIG. 21 provides an amino acid sequence of a variant (SEQ ID NO: 21) of the humanized V_{L} domain of FIG. 5 (SEQ ID NO: 5). This V_{L} amino acid sequence is expressed by clone HX28.

FIG. 22 provides a nucleic acid sequence (SEQ ID NO: 22) encoding the amino acid sequence depicted in FIG. 21. The nucleic acid sequence is present in clone HX28.

FIG. 23 provides an amino acid sequence of a human IgG heavy chain constant region that may be coupled to a humanized V_{H} domain when forming an IgG antibody that specifically binds VEGF-D (SEQ ID NO: 23).

FIG. 24 provides an amino acid sequence of a human IgG light chain constant region that may be coupled to a humanized V_{L} domain when forming an IgG antibody that specifically binds VEGF-D (SEQ ID NO: 24).

FIG. 25 provides an amino acid sequence of a humanized scFv antigen binding fragment that specifically binds VEGF-D comprising the humanized V_{H} domain of FIG. 1, the humanized V_{L} domain of FIG. 5, and a 15 amino acid linker sequence indicated by underlined residues (SEQ ID NO: 25). If there is any doubt, the numbering referred to herein is derived from this reference amino acid sequence (SEQ ID NO: 25). The V_{H} runs from position 1 to position 118. The linker runs from position 119 to position 133. The V_{L} runs from position 134 to position 246. To ensure consistent numbering, the same scFv-based numbering has been used for scFv clones that have been re-formatted into IgG antibodies. For example, the S144G scFv variation refers to an amino acid substitution in the light chain variable region at position 11 in the corresponding IgG antibody: 144 [position on the scFv]−133 [end of the linker preceding the V_{L} region]=position 11 of the IgG light chain.

FIG. 26 provides an amino acid sequence of CDR1 (SEQ ID NO: 26) of the V_{H} domain of FIG. 1 and FIG. 25.

FIG. 27 provides an amino acid sequence of CDR2 (SEQ ID NO: 27) of the V_{H} domain of FIG. 1 and FIG. 25.

FIG. 28 provides an amino acid sequence of CDR3 (SEQ ID NO: 28) of the V_{H} domain of FIG. 1 and FIG. 25.

FIG. 29 depicts two VEGFR-2-Ba/F3 ³H-thymidine incorporation bioassays, as detailed in Example 6, testing VEGF-D neutralizing activity of the humanized IgG antibody derived from clone F61. The inhibitory concentration that achieves 50% inhibition of ³H-thymidine incorporation (IC₅₀) may be calculated from the dose-response curves. The solid circles and solid line represent VEGF-D from Source 1. The solid triangles and broken line represent VEGF-D from Source 2. Cells not stimulated with VEGF-D are represented by solid and broken lines joining test antibody concentrations 0.1 μg/mL and 100 μg/mL.

FIG. 30 depicts the bioassays of FIG. 29 testing the humanized IgG antibody derived from clone HW78.

FIG. 31 depicts the bioassays of FIG. 29 testing the humanized antibody derived from clone HH69.

FIG. 32 depicts in duplicate the bioassays of FIG. 29 testing the humanized IgG antibody derived from clone HH41, except that VEGF-D from Source 2 is represented by solid triangles and solid lines, rather than broken lines.

FIG. 33 depicts the bioassays of FIG. 29 testing the humanized IgG antibody derived from clone HX28.

FIG. 34 depicts the bioassays of FIG. 29 testing the humanized IgG antibody derived from clone HU75.

FIG. 35 depicts the bioassays of FIG. 29 testing the humanized IgG antibody derived from clone DI26.

FIG. 36 depicts the bioassays of FIG. 29 testing the humanized IgG antibody derived from clone AW61.

FIG. 37 depicts the average IC₅₀, calculated from the IC₅₀ of Source 1 and Source 2 VEGF-D dimer, of humanized IgG antibodies derived from clones F61, HW78, HH69, HH41, HX28, HU75, DI26 and AW61 plotted against their corresponding dissociation constant (K_{D}) values.

FIG. 38 depicts one bioassay of FIG. 29 testing the humanized IgG antibody derived from clone F61. The vertical broken line represents the IC₅₀.

FIG. 39 depicts one bioassay of FIG. 29 testing the humanized IgG antibody derived from clone HW78. The vertical broken line represents the IC₅₀.

FIG. 40 depicts one bioassay of FIG. 29 testing the humanized antibody derived from clone HH69. The vertical broken line represents the IC₅₀.

FIG. 41 depicts one bioassay of FIG. 29 testing the humanized IgG antibody derived from clone HH41. The vertical broken line represents the IC₅₀.

FIG. 42 depicts one bioassay of FIG. 29 testing the humanized IgG antibody derived from clone HX28. The vertical broken line represents the IC₅₀.

FIG. 43 depicts one bioassay of FIG. 29 testing the humanized IgG antibody derived from clone HU75. The vertical broken line represents the IC₅₀.

FIG. 44 depicts one bioassay of FIG. 29 testing the humanized IgG antibody derived from clone DI26. The vertical broken line represents the IC₅₀.

FIG. 45 depicts one bioassay of FIG. 29 testing the humanized IgG antibody derived from clone AW61. The vertical broken line represents the IC₅₀.

FIG. 46 provides an amino acid sequence of CDR2 (SEQ ID NO: 29) of the V_{H} domain of FIG. 1 and FIG. 25 comprising the N55S substitution that corresponds to amino acid residue 6 of SEQ ID NO: 27.

FIG. 47 (SEQ ID NO: 30) provides the human VEGF-D polynucleotide sequence.

FIG. 48 (SEQ ID No: 31) provides the human VEGF-D amino acid sequence as encoding by the polynucleotide of FIG. 39.

FIG. 49 (SEQ ID NO: 32) provides the polynucleotide sequence for the forward primer used for PCR amplification of the DNA fragment encoding the extracellular domain of human VEGFR-3.

FIG. 50 (SEQ ID NO: 33) provides the polynucleotide sequence for the reverse primer used for PCR amplification of the DNA fragment encoding the extracellular domain of human VEGFR-3.

FIG. 51 provides an amino acid sequence of CDR1 (SEQ ID NO: 34) of the V_{L} domain of FIG. 5 and FIG. 25.

FIG. 52 provides an amino acid sequence of CDR2 (SEQ ID NO: 35) of the V_{L} domain of FIG. 5 and FIG. 25.

FIG. 53 provides an amino acid sequence of CDR3 (SEQ ID NO: 36) of the V_{L} domain of FIG. 5 and FIG. 25.

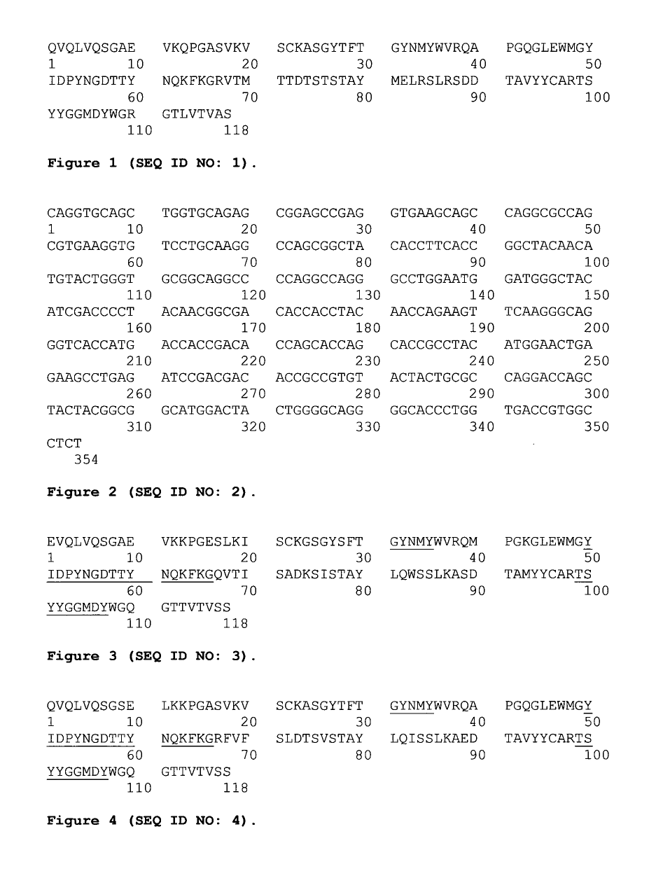

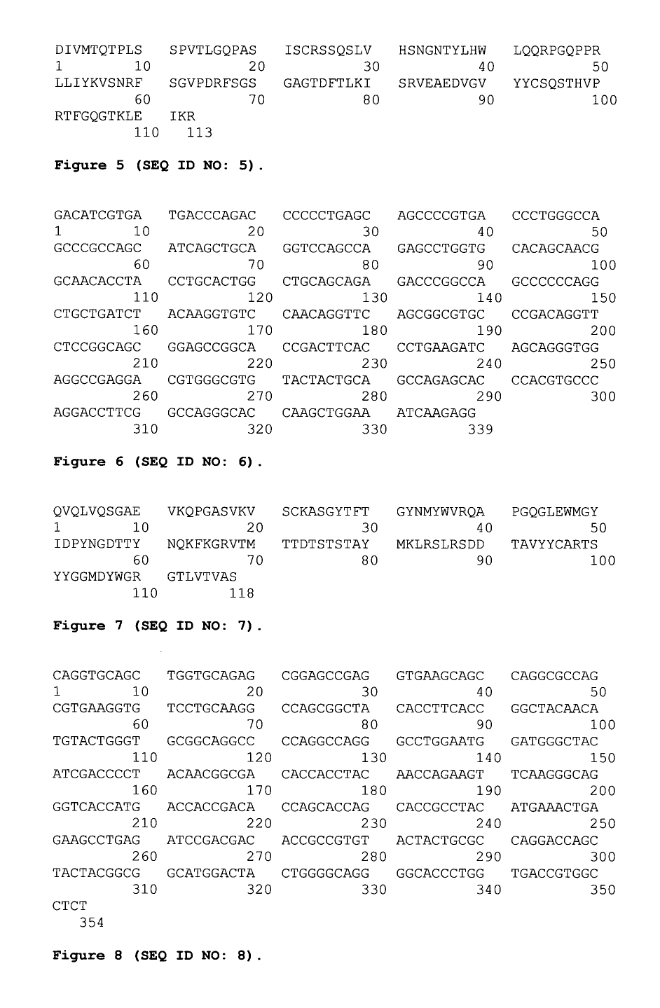

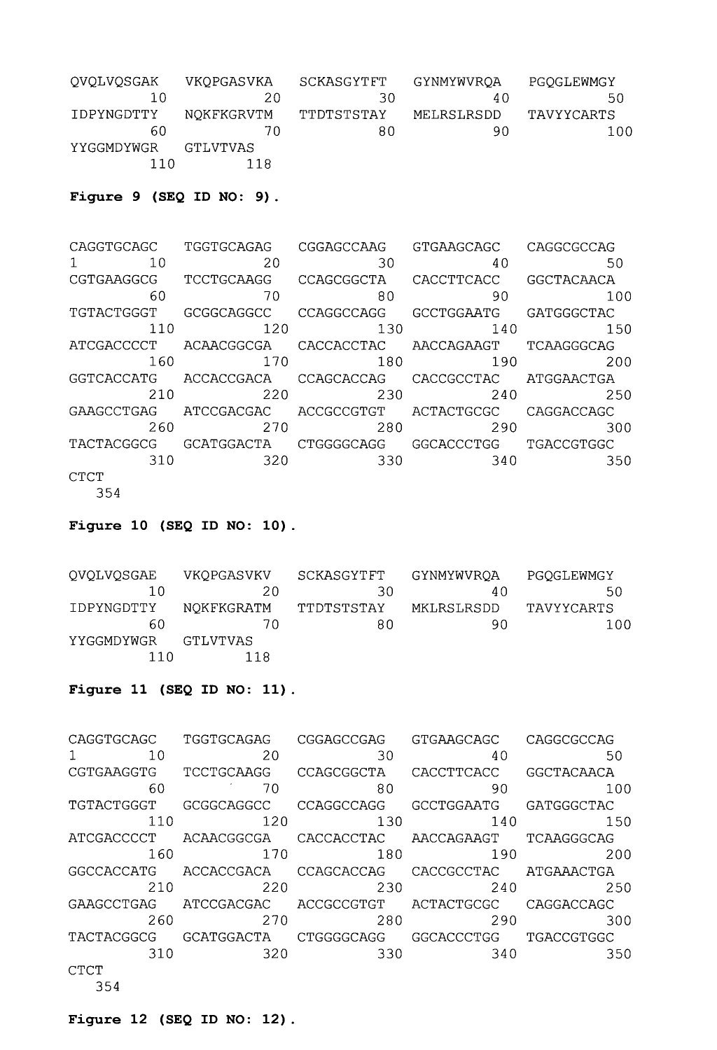

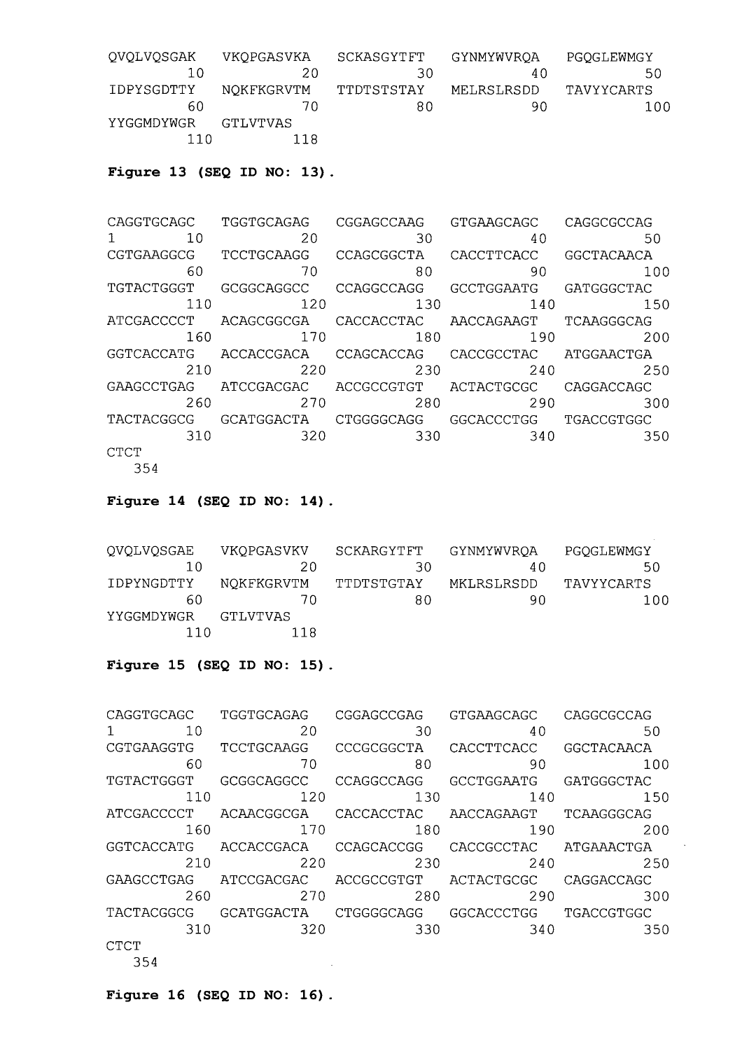

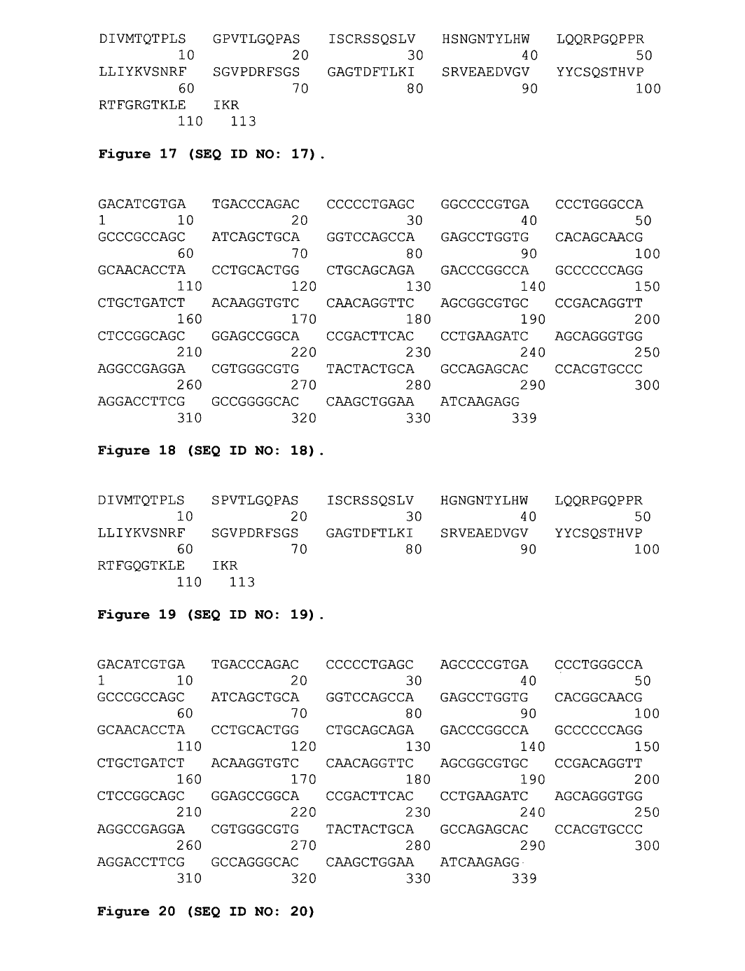

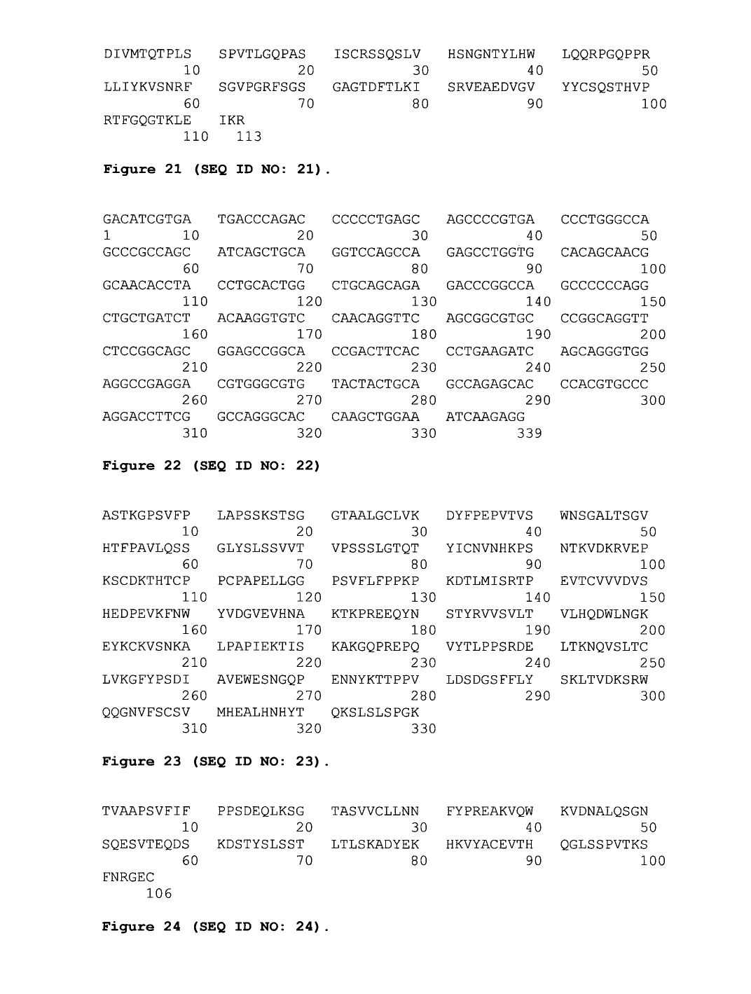

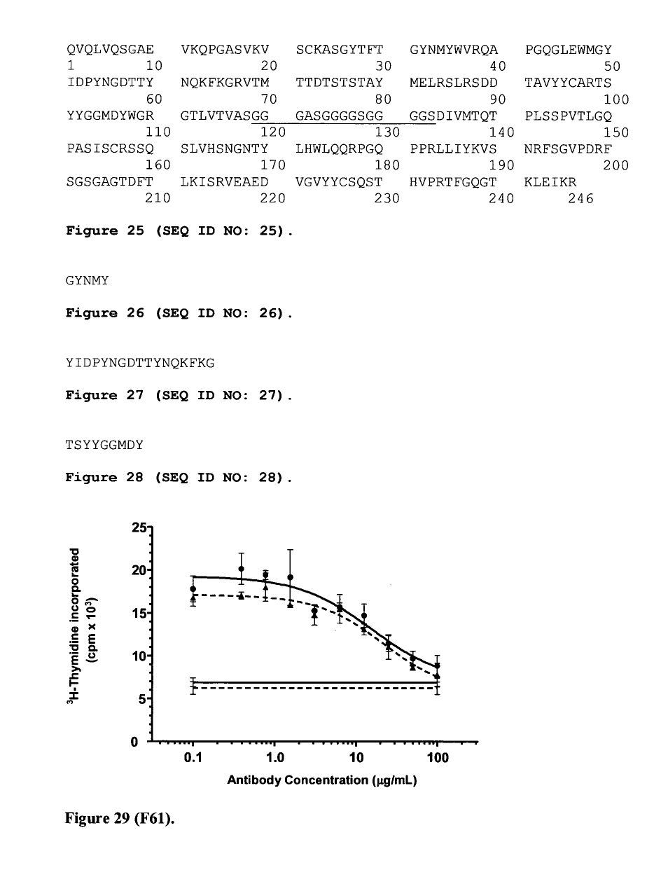

### DETAILED DESCRIPTION

The invention will now be described in detail by way of reference to the following non-limiting examples and figures.

“Lymphangiogenesis” refers to formation of lymphatic vessels, particularly from pre-existing lymphatic vessels.

“Angiogenesis” refers to formation of blood vessels, particularly the proliferation of new capillaries from pre-existing blood vessels.

Importantly, the lymphangiogenesis induced by VEGF-D and VEGF-C promotes metastatic spread of tumor cells to the lymphatic vessels and lymph nodes, and the angiogenesis induced by VEGF-D and VEGF-C in tumors can promote solid tumor growth and metastatic spread. Furthermore, clinicopathological data indicates a role for these growth factors in a range of prevalent human cancers. For example, VEGF-D expression was reported to be an independent prognostic factor for both overall and disease-free survival in colorectal cancer.

“Functional variant” or “variant” as used herein can be used interchangeably and includes either natural amino acid sequence variants or artificially modified amino acid sequence variants that specifically bind and partially or fully block, neutralize, reduce or antagonize a biological activity of VEGF-D. Such artificially modified variants can be made by synthetic chemistry of recombinant DNA mutagenesis techniques that are well known to persons skilled in the art.

The number of amino acid substitutions in the V_{H} domain amino acid sequence provided as SEQ ID NO: 1 to produce a SEQ ID NO: 1 variant is no more than 10. The preferred number of amino acid substitutions in the V_{H} domain amino acid sequence provided as SEQ ID NO: 1 to produce a SEQ ID NO: 1 variant is 1, 2, or 3.

The number of amino acid substitutions in the V_{L} domain amino acid sequence provided as SEQ ID NO: 5 to produce a SEQ ID NO: 5 variant is no more than 10. The preferred number of amino acid substitutions in the V_{L} domain amino acid sequence provided as SEQ ID NO: 5 to produce a SEQ ID NO: 5 variant is 1 or 2.

Suitably, a functional variant of SEQ ID NO: 1 has an amino acid sequence identity of at least 90%, more preferably at least 91%, yet more preferably at least 92%, yet more preferably at least 93% and yet more preferably at least 94% with SEQ ID NO: 1. Suitably, a functional variant of SEQ ID NO: 1 has an amino acid sequence identity of at least 95%, preferably at least 96%, more preferably at least 97% and yet more preferably at least 98% with SEQ ID NO: 1. A functional variant of SEQ ID NO: 1 may have an amino acid sequence identity of at least 99% with SEQ ID NO: 1.

Suitably, a functional variant of SEQ ID NO: 5 has an amino acid sequence identity of at least 90%, more preferably at least 91%, yet more preferably at least 92%, yet more preferably at least 93% and yet more preferably at least 94% with SEQ ID NO: 5. Suitably, a functional variant of SEQ ID NO: 5 has an amino acid sequence identity of at least 95%, preferably at least 96%, more preferably at least 97% and yet more preferably at least 98% with SEQ ID NO: 5. A functional variant of SEQ ID NO: 1 may have an amino acid sequence identity of at least 99% with SEQ ID NO: 5.

In any case, the functional variant will still be capable of specifically binding and partially or fully blocking, neutralizing, reducing or antagonizing a biological activity of VEGF-D.

A functional amino acid sequence variant of these amino acid sequences can be obtained by substitution, replacement, addition, insertion, omission and/ or deletion of an amino acid of these amino acid sequences, and/or a functional nucleic acid sequence for producing said amino acid sequences or functional amino acid sequence variants. In particular, this refers to amino acid sequences comprising conservative substitution without losing their property as a functional variant. The amino acid sequences of the antibody of the invention or their functional variants can also be linked with peptides or polypeptides or with further chemical groups such as glycosyl groups, lipids, phosphates, acetyl groups or the like, provided they do not strongly adversely influence their effect.

As used herein, “substituted” or “substitution” includes substitution, replacement, addition, insertion, omission and/ or deletion of an amino acid residue.

Functional variants of the V_{H} or V_{L} amino acid sequences comprising the antibody of the invention generally may be identified by modifying the amino acid sequence then assaying the resulting antibody for the ability to specifically bind to VEGF-D and/ or partially or fully block, neutralize, reduce or antagonize a biological activity of VEGF-D.

Thus, the specifically stated amino acid sequences can vary, provided individual substitution, addition and/or omission of an amino acid does not strongly impair the function of the antibody, i.e. its ability to specifically bind VEGF-D.

In addition to naturally occurring amino acids, non-naturally occurring amino acids, or modified amino acids, are also contemplated and within the scope of the invention. In fact, as used herein, “amino acid” refers to naturally occurring amino acids, non-naturally occurring amino acids, and amino acid analogs, and to the D or L stereoisomers of each.

Natural amino acids include alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y), valine (V), hydroxyproline (O and/or Hyp), isodityrosine (IDT), and di-isodityrosine (di-IDT). Hydroxyproline, isodityrosine, and di-isodityrosine are formed post-translationally. Use of natural amino acids, in particular the 20 genetically encoded amino acids, is preferred.

The substitutions may be conservative amino acid substitutions, in which the substituted amino acid has similar structural or chemical properties with the corresponding amino acid in the reference sequence. Alternatively, the substitutions may be non-conservative amino acid substitutions.

By way of example, conservative amino acid substitutions involve substitution of one aliphatic or hydrophobic amino acids, e.g., alanine, valine, leucine and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine and histidine, with another; and replacement of one small amino acid, e.g., alanine, serine, threonine, methionine, and glycine, with another.

Antibody variants may be obtained in which a constituent V_{H} or V_{L} amino acid sequence has been chemically modified at the level of amino acid side chains, of amino acid chirality, and/ or of the peptide backbone. These alterations are intended to provide V_{H} and/or V_{L} amino acid sequences having similar or improved therapeutic, diagnostic and/ or pharmacokinetic properties.

For example, when an amino acid sequence is susceptible to cleavage by peptidases following injection into a subject, replacement of a particularly sensitive peptide bond with a non-cleavable peptide bond can provide a peptide more stable and thus more useful as a therapeutic agent. Similarly, the replacement of an L-amino acid residue is a standard way of rendering an amino acid sequence less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life will be known to the person skilled in the art.

The term “constituent V_{H} amino acid sequence” refers to a V_{H} amino acid sequence provided as SEQ ID NO: 1 or a variant thereof which contains from 1 to 10 amino acid substitutions that is included in an antibody that specifically binds VEGF-D.

The term “constituent V_{L} amino acid sequence” refers to a V_{L} amino acid sequence provided as SEQ ID NO: 5 or a variant thereof which contains from 1 to 10 amino acid substitutions that is included in an antibody that specifically binds VEGF-D.

In the case of single-domain antibodies, the antibody essentially consists of the “constituent V_{H} amino acid sequence”.

An “agent” or “therapeutic agent” of the invention as used herein refers to an antibody, including an antigen binding fragment thereof, a nucleic acid molecule, a vector or a pharmaceutical composition according to the invention.

The antibody of the invention may also comprise a non-specific linker that can be adjoined to SEQ ID NO: 1 or a variant thereof, or to SEQ ID NO: 5 or a variant thereof Such a linker is not involved in biological activity. Rather, the linker may serve as a spacer between the amino acid sequence and a functional moiety. One example would be a linker used between the antibody and biotin, where biotin is used for immobilization of the antibody. Other uses for a linker include attachment of a moiety to aid purification or detection. Alternatively, and importantly, a linker may allow attachment of a moiety to the antibody that enables specific delivery of the antibody to a particular target, such as a cell or tissue, spatially or temporally.

VEGF-D Polypeptides

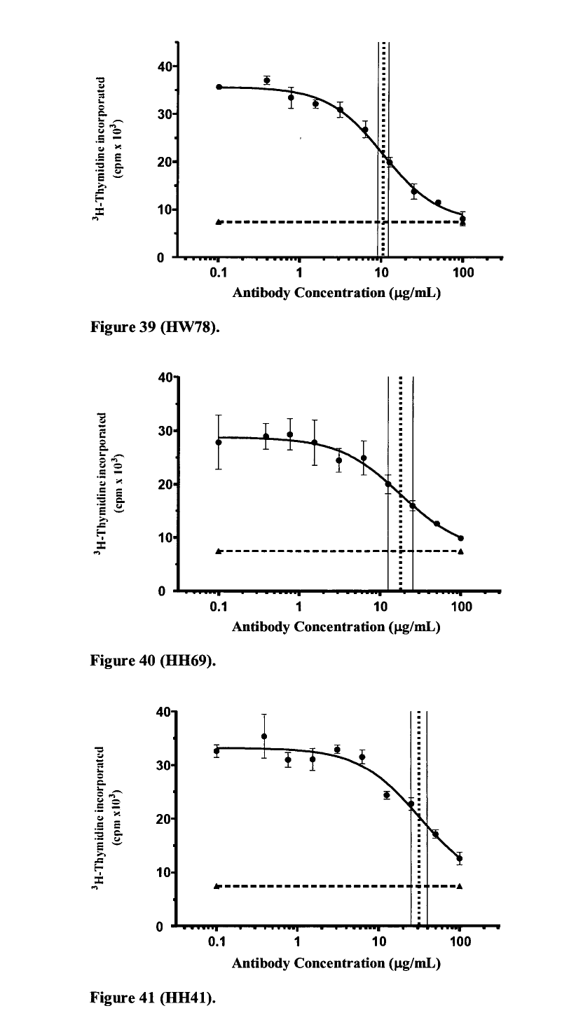

VEGF-D (SEQ ID NOs: 30 and 31; FIGS. 39 and 40) was isolated as described in detail in International Patent Application No. PCT/US97/14696 (WO98/07832) and (Achen et al., Proc. Natl. Acad. Sci. USA 95: 548-553, 1998), both incorporated herein by reference.

VEGF-D is initially expressed as a prepro-peptide that undergoes N-terminal and C-terminal proteolytic processing, and forms non-covalently linked dimers. VEGF-D stimulates mitogenic responses in endothelial cells thru its receptors, VEGFR-3 and VEGFR-2. During embryogenesis, VEGF-D is expressed in a complex temporal and spatial pattern, and its expression persists in the heart, lung, and skeletal muscles in adults.

The prepro-VEGF-D polypeptide has a putative signal peptide of 21 amino acids and is apparently proteolytically processed in a manner analogous to the processing of prepro-VEGF-C. A “recombinantly matured” VEGF-D, VEGF-D ANAC, containing amino acid residues 93 to 201, and lacking residues 1-92 and 202-354 of SEQ ID NO: 31, retains the ability to activate receptors VEGFR-2 and VEGFR-3, and appears to associate as non-covalently linked dimers.

Preparation of Amino Acid Sequences and Variants

The constituent V_{H} or V_{L} amino acid sequences and antibodies of the present invention comprising constituent V_{H} or V_{H} and V_{L} amino acid sequences can be prepared in any suitable manner. For example, polypeptides can be produced recombinantly, synthetically or by a combination of these methods. Means for preparing such peptides are well understood in the art.

Polypeptides may be synthesized using commercially available peptide synthesizers, for example by solid phase synthesis, or may also be produced using cell-free translation systems and RNA molecules derived from DNA constructs that encode the amino acid sequences. Alternatively, the polypeptide is made by transfecting host cells with expression vectors that comprise a DNA sequence that encodes the respective amino acid sequence and then inducing expression of the polypeptide in the host cells.

For recombinant production, a recombinant construct comprising a sequence which encodes the amino acid sequence, or a variant thereof, is introduced into host cells by conventional methods such as calcium phosphate transfection, DEAE-dextran mediated transfection, transvection, microinjection, cationic lipid-mediated transfection, electroporation, transduction, scrape lading, ballistic introduction or infection. An example of a genetic engineering production method is manipulation of microorganisms such as E. coli. These are manipulated so that they express the amino acid sequences.

Polypeptides comprising the V_{H} or V_{H} and V_{L} amino acid sequence, or a variant thereof, may be expressed in suitable host cells, such as for example, mammalian cells, yeast, bacteria, insect cells or other cells under the control of appropriate promoters using conventional techniques. Suitable hosts include, but are not limited to, E. coli, P. pastoris, COS cells, and 293 HEK cells. Following transformation of the suitable host strain and growth of the host strain to an appropriate cell density, the cells are harvested by centrifugation, disrupted by physical or chemical means, and the resulting crude extract retained for further purification of the peptide, or variant thereof.

Purification of Recombinantly Expressed Peptides

Conventional procedures for isolating recombinant proteins from transformed host cells, such as isolation by initial extraction from cell pellets or from cell culture medium, such as acid extraction, followed by salting-out, such as ammonium sulphate or ethanol precipitation, and one or more chromatography steps may be used to recover and isolate the recombinant V_{H} or V_{H} and V_{L} amino acid sequence or antibody comprising the constituent V_{H} or V_{H} and V_{L} amino acid sequence from recombinant cell cultures. Such chromatography may include aqueous anion or cation exchange chromatography, size exclusion chromatography, high pressure liquid chromatography, affinity chromatography, phosphocellulose chromatography, hydrophobic interaction chromatography, hydroxyapatite chromatography, or lectin chromatography. Well known techniques for refolding proteins may be employed to regenerate active conformation when the peptide is denatured during isolation and or purification.

To produce a glycosylated amino acid sequence, or variant thereof, it is preferred that recombinant techniques be used. To produce a glycosylated peptide, or variant thereof, it is preferred that mammalian cells such as, COS-7 and Hep-G2 cells be employed in the recombinant techniques.

Nucleic Acid Molecules

The terms “nucleic acid molecule”, “nucleic acid”, “nucleic acid sequence”, and “functional nucleic acid molecule” are used interchangeably herein and refer to any nucleic acid molecule, DNA or RNA, which encodes a corresponding constituent V_{H} or V_{L} amino acid sequence of an antibody according to the invention. These DNA or RNA molecules can also be present in vectors.

The present invention also provides isolated nucleic acid molecules that encode constituent V_{H} or V_{H} and V_{L} amino acid sequences as well as antibodies of the invention comprising constituent V_{H} or V_{H} and V_{L} amino acid sequences. The present nucleic acid molecules also encompass nucleic acid molecules having sequences that are capable of hybridizing under stringent conditions, preferably highly stringent conditions, to the nucleic acid molecules that encode the constituent V_{H} or V_{H} and V_{L} amino acid sequences. The person skilled in the art will appreciate that hybridization conditions are based on the melting temperature (Tm) of the nucleic acid molecule binding complex or probe.

The term “stringent conditions”, as used herein, is the “stringency” which occurs within a range from about Tm-5 to about Tm-20 (about 5° C. to about 20° C. below the melting temperature of the probe).

As used herein, “highly stringent” conditions employ at least 0.2×SSC buffer and at least 65° C. As recognized in the art, stringency conditions can be attained by varying a number of factors of the hybridization solution such as the length and nature, i.e., DNA or RNA, of the probe; the length and nature of the target sequence; the concentration of the salts; and the concentration of other components, such as formamide, dextran sulfate, and polyethylene glycol. All of these factors may be varied to generate conditions of stringency which are equivalent to the conditions listed above.

Nucleic acid molecules comprising sequences encoding a constituent V_{H} or V_{H} and V_{L} amino acid sequence may be synthesized in whole or in part using chemical methods or recombinant methods which are known in the art.

The nucleic acid molecules are useful for producing constituent V_{H} or V_{H} and V_{L} amino acid sequences and therefore an anti-VEGF-D antibody of the invention. For example, an RNA molecule encoding a peptide may be used in a cell-free translation system or in a host cell to prepare such peptide.

The nucleic acid molecule encoding a constituent V_{H} or V_{H} and V_{L} amino acid sequence of an antibody of the invention can be used to express a recombinant peptide using techniques well known in the art.

The nucleic acid molecule of the invention also relates to a DNA sequence that can be derived from the amino acid sequence of an antibody of the invention bearing in mind the degeneracy of codon usage. This is well known in the art, as is knowledge of codon usage in different expression hosts, which is helpful in optimizing the recombinant expression of the constituent V_{H} or V_{H} and V_{L} amino acid sequence or an antibody of the invention comprising the constituent V_{H} or V_{H} and V_{L} amino acid sequence.

The invention also provides nucleic acid molecules which are complementary to all the above described nucleic acid molecules.

When the nucleic acid molecules of the invention are used for the recombinant production of a constituent V_{H} or V_{H} and V_{L} amino acid sequence of an antibody of the present invention, the nucleic acid molecule may include the coding sequence for the amino acid sequence by itself or the coding sequence for the amino acid sequence in reading frame with other coding sequences, such as those encoding a leader or secretory sequence, a pre-, or pro-, or prepro-protein sequence, or other fusion peptide portions. For example, a marker sequence which facilitates purification of the fused peptide can be encoded. In certain embodiments of this aspect of the invention, the marker sequence is a hexa-histidine peptide, as provided in the pQE vector (Qiagen, Inc.), or is an HA tag, or is glutathione-S-transferase.

The nucleic acid molecule may also contain non-coding 5′ and 3′ sequences, such as transcribed, non-translated sequences, splicing and polyadenylation signals, ribosome binding sites and sequences that stabilize mRNA.

Vectors, Host Cells, Expression

The present invention also relates to vectors which comprise a nucleic acid molecule of the present invention, and host cells which are genetically engineered with vectors of the invention and to the production of constituent V_{H} or V_{H} and V_{L} amino acid sequence or an antibody of the invention comprising the constituent V_{H} or V_{H} and V_{L} amino acid sequence by recombinant techniques.

A nucleic acid molecule encoding a constituent V_{H} or V_{H} and V_{L} amino acid sequence or encoding an antibody of the invention comprising a constituent V_{H} or V_{H} and V_{L} amino acid sequence may be introduced into an expression vector and used to transform cells. Suitable expression vectors include, for example, chromosomal, non-chromosomal, episomal, virus-derived systems and synthetic DNA sequences, e.g., transposons, insertion elements, bacterial plasmids, phage DNAs, yeast plasmids, yeast episomes, yeast chromosomal elements, vectors derived from combinations of plasmids and phage DNAs, bacteriophages, viral DNA such as derivatives of SV40, vaccinia, adenovirus, papova viruses, fowl pox virus, pseudorabies, baculovirus, and retrovirus, and vectors derived from combinations thereof, such as those derived from plasmid and bacteriophage genetic elements, such as cosmids and phagemids. The DNA sequence may be introduced into the expression vector by conventional procedures known in the art.

The expression systems may contain control regions that regulate as well as engender expression.

In the expression vector, the DNA sequence which encodes the amino acid sequence is operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator.

The recombinant expression vectors may also include an origin of replication and a selectable marker, such as the ampicillin resistance gene of E. coli to permit selection of transformed cells, i.e., cells that are expressing the heterologous DNA sequences. The nucleic acid molecule encoding the constituent V_{H} or V_{L} amino acid sequence of an antibody, or an antibody per se, may be incorporated into the vector in frame with translation initiation and termination sequences.

Cell-free translation systems can also be employed to produce such proteins using RNAs derived from the DNA constructs of the present invention.

For recombinant production, host cells can be genetically engineered to incorporate expression systems or portions thereof for nucleic acid molecules of the present invention. Introduction of nucleic acid molecules into host cells can be effected by one skilled in the art using methods such as calcium phosphate transfection, DEAE-dextran mediated transfection, transfection, microinjection, cationic lipid-mediated transfection, electroporation, transduction, scrape loading, ballistic introduction or infection.

Representative examples of appropriate hosts include bacterial cells, such as meningococci, streptococci, staphylococci, E. coli, Streptomyces and Bacillus subtilis cells; fungal cells and Aspergillus cells; yeast cells; insect cells such as Drosophila S2 and Spodoptera Sf9 cells; animal cells such as CHO, COS, HeLa, C127, 3T3, BHK, HEK293 and Bowes melanoma cells; and plant cells.

Generally, any system or vector suitable to maintain, propagate or express nucleic acid molecules to produce an amino acid sequence in a host may be used. The appropriate nucleic acid sequence may be inserted into an expression system by any of a variety of well known and routine techniques.

For secretion of the translated amino acid sequence into the lumen of the endoplasmic reticulum, into the periplasmic space, or into the extracellular environment, appropriate secretion signals may be incorporated into the desired amino acid sequence. These signals may be endogenous to the amino acid sequence or they may be heterologous signals.

Additional exemplary expression construct include a virus or engineered construct derived from a viral genome. The expression construct generally comprises a nucleic acid encoding the gene or binding construct, including any nucleic acid molecule described herein, to be expressed and also additional regulatory regions that will effect the expression of the gene in the cell to which it is administered. Such regulatory regions include for example promoters, enhancers, polyadenylation signals and the like.

DNA may be introduced into a cell using a variety of viral vectors. In such embodiments, expression constructs comprising viral vectors containing the genes of interest may be adenoviral (see, for example, U.S. Pat. No. 5,824,544; U.S. Pat. No. 5,707,618; U.S. Pat. No. 5,693,509; U.S. Pat. No. 5,670,488; U.S. Pat. No. 5,585,362, each incorporated herein by reference), retroviral (see, for example, U.S. Pat. No. 5,888,502; U.S. Pat. No. 5,830,725; U.S. Pat. No. 5,770,414; U.S. Pat. No. 5,686,278; U.S. Pat. No. 4,861,719, each incorporated herein by reference), adeno-associated viral (see, for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479, each incorporated herein by reference), an adenoviral-adenoassociated viral hybrid (see, for example, U.S. Pat. No. 5,856,152 incorporated herein by reference) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688, each incorporated herein by reference) vector. Other vectors described herein may also be employed.

In other embodiments, non-viral delivery is contemplated. These include calcium phosphate precipitation (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990)), DEAE-dextran (Gopal, Mol. Cell Biol., 5:1188-1190 (1985)), electroporation (Tur-Kaspa, et al., Mol. Cell Biol., 6:716-718, (1986); Potter, et al., Proc. Nat. Acad. Sci. USA, 81:7161-7165, (1984)), direct microinjection (Harland and Weintraub, J. Cell Biol., 101:1094-1099 (1985)), DNA-loaded liposomes (Nicolau and Sene, Biochim. Biophys. Acta, 721:185-190 (1982) (Radler, et al., Science, 275(5301):810-4, (1997)); Fraley, et al., Proc. Natl. Acad. Sci. USA, 76:3348-3352 (1979); Feigner, Sci. Am., 276(6):102-6 (1997); Feigner, Hum. Gene Ther., 7(15):1791-3, (1996)), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990)), receptor-mediated transfection (Wu and Wu, J. Biol. Chem., 262:4429-4432 (1987); Wu and Wu, Biochemistry, 27:887-892 (1988); Wu and Wu, Adv. Drug Delivery Rev., 12:159-167 (1993)), and injection of naked DNA (Dubensky, et al., Proc. Nat. Acad. Sci. USA, 81:7529-7533 (1984)) (Benvenisty and Neshif, Proc. Nat. Acad. Sci. USA, 83:9551-9555 (1986)).

Other vector delivery systems that can be employed to deliver a nucleic acid encoding a therapeutic gene into cells include receptor-mediated delivery vehicles. Receptor-mediated gene targeting vehicles generally consist of two components: a cell receptor-specific ligand and a DNA-binding agent. Several ligands have been used for receptor-mediated gene transfer, including asialoorosomucoid (ASOR) (Wu and Wu (1987), supra), transferrin (Wagner, et al., Proc. Nat'l. Acad Sci. USA, 87(9):3410-3414 (1990)), a synthetic neoglycoprotein, which recognizes the same receptor as ASOR, (Ferkol, et al., FASEB. J., 7:1081-1091 (1993); Perales, et al., Proc. Natl. Acad. Sci., USA 91:4086-4090 (1994)) and epidermal growth factor (EGF) (Myers, EPO 0273085).

The vector/expression construct may optionally contain elements such as a 5′ flanking sequence, an origin of replication, a transcription termination sequence, a selectable marker sequence, a ribosome binding site, a signal sequence, and one or more intron sequences. The 5′ flanking sequence may be homologous (i.e., from the same species and/or strain as the host cell), heterologous (i.e., from a species other than the host cell species or strain), hybrid (i.e., a combination of 5′ flanking sequences from more than one source), synthetic, or it may be the native polypeptide 5′ flanking sequence.

Promoters for gene therapy include cytomegalovirus (CMV) promoter/enhancer, long terminal repeat (LTR) of retroviruses, keratin 14 promoter, and a myosin heavy chain promoter.

In a further embodiment, the expression construct comprises a signal sequence, such as a peptide or nucleotide sequence encoding a secretory signal amino acid sequence that acts to direct the secretion of a mature polypeptide or protein from a cell. Exemplary signal sequences for use in gene therapy vectors are well-known in the art.

Antibodies

The term “antibody” is used in the broadest sense and specifically covers, for example, polyclonal antibodies, monoclonal antibodies (including antagonist and neutralizing antibodies), antibody compositions with polyepitopic specificity, and single chain antibodies. The term “antibody” also covers fragments of antibodies (i.e. antibody fragments), provided that they exhibit the desired biological or immunological activity.

“Antibody fragments” comprise a portion of an antibody, preferably the antigen binding or variable region of the intact antibody. Antigen binding fragments retain the same antigen binding specificity and the same or similar neutralizing ability as the antibody from which the fragment was derived.

In certain embodiments of the invention there is provided antigen binding fragments which neutralize the activity of VEGF-D. Such fragments may be functional antigen binding fragments of intact and/or humanized and/or chimeric antibodies such as Fab, Fab′, F(ab′)2, Fv, ScFv fragments of the antibodies described. Also included are single-domain “antibodies”.

Traditionally such fragments are produced by the proteolytic digestion of intact antibodies, for example by papain digestion, but may be produced directly from recombinantly transformed host cells. In addition, antigen binding fragments may be produced using a variety of engineering techniques. Fv fragments appear to have lower interaction energy of their two chains than Fab fragments. To stabilize the association of the V_{H} and V_{L} domains, they have been linked with peptides, disulphide bridges and “knob in hole” mutations.

As used herein, “antibody” specifically includes both an immunoglobulin and any functional fragment that can be derived therefrom.

The term “specific binding”, “specifically binds”, “binds specifically” or “specific for” refers to binding where a molecule binds to a particular epitope on a particular polypeptide without substantially binding to any other polypeptide or polypeptide epitope. Such binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

As used herein, “specific binding” is used in relation to the interaction between a constituent V_{H} or V_{H} and V_{L} amino acid sequence of an antibody, or an antibody of the invention comprising a constituent V_{H} or V_{H} and V_{L} amino acid sequence and VEGF-D polypeptide.

More particularly, “specific binding” is used in relation to the interaction with mature VEGF-D polypeptide, i.e. VEGF-D in which the prepro-, full-length polypeptide has been proteolytically processed at both the N- and C-terminals to produce a mature active VEGF-DΔNΔC protein of approximately 21 kDa (as a monomer) and approximately 110 to 115 amino acid residues in length, and that self-associates as a dimer. For purposes of antibody binding to “mature VEGF-D,” the term “mature VEGF-D” is meant to refer to an N- and C-terminally processed VEGF-DΔNΔC as described immediately above that (a) is a fragment of full length VEGF-D within (and optionally including) amino acids 91 to 205 of SEQ ID NO: 31; (b) that self associates as a dimmer; and (c) that is biologically active as assayed by the Ba/F3 assay described herein (Other forms of biologically active N- and C-terminally processed VEGF-D may exist that are larger or smaller by virtue of having different N- or C-terminal residues, that may be referred to as mature VEGF-D in other contexts.) Exemplary mature VEGF-D forms are described in International Patent Application No. PCT/US97/14696 (WO98/07832) and (Achen et al., Proc. Natl. Acad. Sci. USA 95: 548-553, 1998), both incorporated herein by reference.

“Specific binding” is also used in relation to an agent of the invention that partially or fully blocks, neutralizes, reduces or antagonizes a biological activity of VEGF-D.

“Specific binding” as used herein indicates that an antibody of the invention is incapable of binding or neutralizing VEGF-C to any substantial degree.

In particular, specific binding refers to a molecule having a dissociation constant (K_{D}) at least 2-fold greater than that of a non-specific target, preferably a molecule having a K_{D} at least 4-fold, 6-fold, 8-fold, 10-fold, or greater than that of a non-specific target. Alternatively, specific binding can be expressed as a molecule having a K_{D} for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10^{31 9} M, alternatively at least about 10⁻¹⁰ M, alternatively at least about 10⁻¹¹ M, alternatively at least about 10⁻¹² M, or less. In certain embodiments, an antibody as described herein binds to VEGF-D with an affinity K_{d} ranging from about 10⁻⁸ M to 10⁻¹² M, or about 10⁻⁹ M to 10⁻¹² M, or about 10⁻⁹ M to 10⁻¹¹ M. Affinity is measured using techniques well-known in the art, including but not limited to, surface plasmon resonance, or techniques described in the Examples.

The antibody of the invention may be used in analyzing VEGF-D protein.

Techniques for the production of single chain antibodies can used to produce an antibody of this invention. Also, transgenic mice, or other organisms including other mammals, may be used to express an antibody of the invention.

An “isolated antibody” is one which has been identified and separated and/ or recovered from a component of its natural environment. Contaminant components of its natural environment are materials which would interfere with diagnostic or therapeutic uses for the antibody, and may include enzymes, hormones, and other proteinaceous or non-proteinaceous solutes. Generally, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS PAGE under reducing or non-reducing conditions using Coomassie blue or, preferably, silver stain. An isolated antibody includes the antibody in situ within recombinant cells since at least one component of the antibody's natural environment will not be present. Ordinarily, however, an isolated antibody will be prepared by at least one purification step.

Where antibody fragments are used, the smallest inhibitory fragment that specifically binds to the binding domain of the target protein is preferred.

Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

The modifier “monoclonal” is not to be construed as requiring production of the antibody by any particular method. For preparation of monoclonal antibodies, any technique which provides antibodies produced by continuous cell line cultures can be used. Examples include the hybridoma technique, the trioma technique, the human B-cell hybridoma technique, and the EBV-hybridoma technique.

DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

Monoclonal antibodies or antibody fragments can be isolated from antibody phage libraries. High affinity (nM range) human antibodies can be generated by chain shuffling, as well as combinatorial infection and in vivo recombination as a strategy for constructing very large phage libraries. Thus, these techniques are viable alternatives to traditional monoclonal antibody hybridoma techniques for isolation of monoclonal antibodies.

Phage display technology can be used to produce human antibodies and antibody fragments in vitro, from immunoglobulin variable (V) domain gene repertoires from unimmunized donors. Phage display can be performed in a variety of formats. Several sources of V-gene segments can be used for phage display.

The DNA that encodes the antibody may be modified to produce chimeric or fusion antibody polypeptides. The monoclonal antibodies used herein include “chimeric” antibodies in which a portion of the heavy and/ or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

Antibody Fragments

“Antibody fragments” comprise a portion of an antibody, preferably the antigen binding or variable region of the intact antibody. Examples of antibody fragments include Fab, Fab′, F(ab′)₂, and Fv fragments; diabodies; linear antibodies; single chain antibody molecules (e.g. camelids, shark IgNARs); and multispecific antibodies formed from antibody fragments.

Papain digestion of antibodies produces two identical antigen binding fragments, called “Fab” fragments, and a residual “Fc” fragment, a designation reflecting the ability to crystallize readily. Each Fab fragment is monovalent with respect to antigen binding, i.e., it has a single antigen binding site. Pepsin treatment of an antibody yields a single large F(ab′)₂ fragment which roughly corresponds to two disulfide linked Fab fragments having divalent antigen binding activity and is still capable of cross linking antigen. Fab′ fragments differ from Fab fragments by having additional residues at the carboxy terminal of the C_{H}1 domain including one or more cysteines from the antibody hinge region. Fab′ SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen recognition binding site. This fragment consists of a dimer of one heavy and one light chain variable region domain in tight, non covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) can have the ability to recognize and bind antigen. Such an antibody fragment is typically referred to as a “single-domain antibody”.

“Single chain Fv” abbreviated as “scFv” are antibody fragments that comprise the V_{H} and V_{L} antibody domains connected into a single polypeptide chain. Preferably, the scFv polypeptide further comprises a polypeptide linker between the V_{H} and V_{L} domains which enables the scFv to form the desired structure for antigen binding.

ScFv fragments can be produced by methods well known to those skilled in the art. ScFv may be produced in bacterial cells such as E. coli but are more preferably produced in eukaryotic cells. Disadvantages of ScFv include the monovalency of the product, which precludes an increased avidity due to polyvalent binding, and their short half-life. Attempts to overcome these problems include bivalent (ScFv′)₂, produced from ScFV, containing an additional C terminal cysteine by chemical coupling or by spontaneous site-specific dimerization of ScFv containing an unpaired C terminal cysteine residue.

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues also may be used.

Other techniques for the production of antibody fragments will be apparent to the skilled practitioner. The antibody of choice is a scFv fragment. Fv and scFv are the only species with intact combining sites that are devoid of constant regions; thus, they are suitable for reduced nonspecific binding during in vivo use. The antibody fragment may also be a “linear antibody”, which may be monospecific or bispecific.

Bispecific and Multivalent Antibodies

Bispecific antibodies are antibodies that have binding specificities for at least two different epitopes. Bispecific antibodies can be prepared as full length antibodies or antibody fragments (e.g., F(ab′)₂ bispecific antibodies).

ScFv can be forced to form multimers by shortening the peptide linker to 3 to 12 residues to form “diabodies”. The term “diabodies” refers to small antibody fragments prepared by constructing scFv fragments with short linkers (about 5 to 10 residues) between the V_{H} and V_{L} domains such that inter chain but not intra chain pairing of the V domains is achieved, resulting in a bivalent fragment, i.e., fragment having two antigen binding sites. Bispecific diabodies are heterodimers of two “crossover” scFv fragments in which the V_{H} and V_{L} domains of the two antibodies are present on different polypeptide chains.

According to an alternative “diabody” technology for making bispecific antibody fragments, the fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of scFv dimers has also been reported.

Reducing the linker still further can result in ScFV trimers (“triabodies”) and tetramers (“tetrabodies”). Construction of bivalent ScFV molecules can also be achieved by genetic fusion with protein dimerizing motifs to form “miniantibodies” and “minibodies”. ScFv-ScFv tandems ((ScFv)₂) may also be produced by linking two ScFv units by a third peptide linker. Bispecific diabodies can be produced through the non-covalent association of two single chain fusion products consisting of V_{H} domain from one antibody connected by a short linker to the V_{L} domain of another antibody. The stability of such bispecific diabodies can be enhanced by the introduction of disulphide bridges or “knob in hole” mutations or by the formation of single chain diabodies (ScDb) wherein two hybrid ScFv fragments are connected through a peptide linker. Tetravalent bispecific molecules are available for example by fusing a ScFv fragment to the C_{H}3 domain of an IgG molecule or to a Fab fragment through the hinge region. Alternatively, tetravalent bispecific molecules have been created by the fusion of bispecific single chain diabodies. Smaller tetravalent bispecific molecules can also be formed by the dimerization of either ScFv-ScFv tandems with a linker containing a helix-loop-helix motif (DiBi miniantibodies) or a single chain molecule comprising four antibody variable domains (V_{H} and V_{L}) in an orientation preventing intramolecular pairing (tandem diabody). Bispecific F(ab′)₂ fragments can be created by chemical coupling of Fab′ fragments or by heterodimerization through leucine zippers.

Also available are isolated V_{H} and V_{L} domains, which are known as domain antibodies or “dAb”, which are capable of binding to an antigen.

Bispecific antibodies include cross-linked or “heteroconjugate” antibodies. Heteroconjugate antibodies are composed of two covalently joined antibodies. It is contemplated that the antibodies may be prepared in vitro using known methods in synthetic protein chemistry, including those involving crosslinking agents. For example, one of the antibodies in the heteroconjugate can be coupled to avidin, the other to biotin. Heteroconjugate antibodies may be made using any convenient cross-linking methods. Suitable cross-linking agents and cross-linking techniques are well known in the art.

Methods for making bispecific antibodies are known in the art. Traditional production of full length bispecific antibodies is based on the co-expression of two immunoglobulin heavy chain-light chain pairs, where the two chains have different specificities.

According to a different approach, antibody variable domains with the desired binding specificity (antibody-antigen combining sites) are fused to immunoglobulin constant domain sequences. For example, bispecific antibodies can be prepared using chemical linkage.

Recent progress has facilitated the direct recovery of Fab′-SH fragments from E. coli, which can be chemically coupled to form bispecific antibodies. Various techniques for making and isolating bispecific antibody fragments directly from recombinant cell culture have also been described.

Antibodies with more than two valencies are contemplated for use in the invention. For example, trispecific antibodies can be prepared. Exemplary epitopes for bispecific antibodies and trispecific antibodies of the invention include additional VEGF-D epitopes and epitopes from other VEGF family members (VEGF-A, -B, -C; PDGF-A, -B, -C, and -D). See, e.g., U.S. Patent Publication No. 2005/0282233, Eriksson et al., incorporated herein by reference in its entirety.

A multivalent antibody may be internalized (and/ or catabolized) faster than a bivalent antibody by a cell expressing an antigen to which the antibodies bind. The antibodies used in the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight antigen binding sites. The multivalent antibody comprises at least one polypeptide chain (and may comprise two polypeptide chains), wherein the polypeptide chain(s) comprise two or more variable domains. For instance, the polypeptide chain(s) may comprise VD₁-(X₁)ₙ-VD₂-(X₂)ₙ-Fc, wherein VD₁ is a first variable domain, VD₂ is a second variable domain, Fc is one polypeptide chain of an Fc region, X₁ and X₂ represent an amino acid or polypeptide, and n is 0 or 1. For instance, the polypeptide chain(s) may comprise: V_{H}-C_{H}1-flexible linker-V_{H}-C_{H}1-Fc region chain; or V_{H}-C_{H}1-V_{H}-C_{H}1-Fc region chain. The multivalent antibody herein may, for instance, comprise from about two to about eight light chain variable domain polypeptides. The light chain variable domain polypeptides contemplated here comprise a light chain variable domain and, optionally, further comprise a C_{L} domain.

Such antibodies according to the invention also may be employed in analytical or diagnostic methods to detect peptides, polypeptides or proteins, of variants or fragments thereof.

Composition for Administration

The antibody or other agent of the invention may be provided as a pharmaceutical composition or veterinary composition.

A “pharmaceutical composition” is one which is suitable for administration to humans. A “veterinary composition” is one that is suitable for administration to animals.

The pharmaceutical or veterinary compositions used in the methods of the invention may comprise a pharmaceutically acceptable carrier and optionally another therapeutic agent. Each carrier, diluent, adjuvant and/or excipient must be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained.

Similarly, a “pharmaceutically acceptable” salt or ester is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

The pharmaceutical composition may be administered orally, topically, or parenterally in formulations containing conventional non-toxic pharmaceutically acceptable carriers, adjuvants, and vehicles.

The term “parenteral” as used herein includes intravenous, intraarterial, intraperitoneal, intramuscular, subcutaneous, subconjunctival, intracavity, transdermal and subcutaneous injection, aerosol for administration to lungs or nasal cavity or administration by infusion by, for example, osmotic pump.

The agent of the invention may be directed against angiogenesis associated with VEGF-D, particularly angiogenesis of cancerous diseases. Alternatively, the agent may be directed against a condition responsive to neutralizing VEGF-D. The condition may include angiogenesis, particularly dysregulated angiogenesis, dysregulated lymphangiogenesis, rheumatoid arthritis, psoriasis, lymphangiolieomyomatosis, and other inflammatory conditions.

Pharmaceutical Compositions

Pharmaceutical compositions may comprise a nucleic acid molecule, a vector, or an antibody of the invention. Preferably, a pharmaceutical composition comprises an antibody of the invention. Such pharmaceutical compositions generally comprise further a pharmaceutically acceptable carrier, excipient, or diluent.

Veterinary Compositions

A nucleic acid molecule, a vector, or an antibody of the invention may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue, particularly adapted for protection through the rumen if to be administered to ruminants;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, e.g. as a cream, ointment or spray applied to the skin; or

(d) intravaginally, e.g. as a pessary, cream or foam.

An agent of the invention can be administered in different ways, for example intravenously, subcutaneously or orally in capsule or tablet form. If the agent contains a nucleic acid molecule, administration can also be done using an ex vivo procedure, which comprises removal of cells from an organism, penetration of the nucleic acid molecule into these cells, and repenetration of the treated cells into the organism.

Carriers and Excipients

Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or antibodies; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

Oral excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Although any suitable carrier known to those of ordinary skill in the art may be employed in the pharmaceutical compositions of this invention, the type of carrier will vary depending on the mode of administration and whether a substantial release is desired. For parenteral administration, such as subcutaneous injection, the carrier preferably comprises water, saline, alcohol, a fat, a wax, or a buffer.

Biodegradable microspheres (e.g., polylactic galactide) may also be employed as carriers for the pharmaceutical compositions of this invention. Optionally, the pharmaceutical composition comprises an adjuvant.

The antibody, nucleic acid molecule, vector, or pharmaceutical composition of the invention may be administered orally as tablets, aqueous or oily suspensions, lozenges, troches, powders, granules, emulsions, capsules, syrups or elixirs. The composition for oral use may contain an agent selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

Compositions for parenteral administration include sterile aqueous or non-aqueous solutions, suspensions, and emulsions. Aqueous and non-aqueous sterile injection solutions may contain anti-oxidants, buffers, bacteriostats, and solutes, which render the composition isotonic with the blood of the recipient, and aqueous and non-aqueous sterile suspensions, which may include suspending agents or thickening agents.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The pharmaceutical composition may also contain other active compounds providing supplemental, additional, or enhanced therapeutic functions.

Other therapeutically useful agents, such as growth factors (e.g., BMPs, TGF-P, FGF, IGF), cytokines (e.g., interleukins and CDFs), antibiotics, and any other therapeutic agent beneficial for the condition being treated may optionally be included in or administered simultaneously or sequentially with the agent of the invention. Other agents that may be effective for those purposes include tumor necrosis factor (TNF), an antibody capable of inhibiting or neutralizing the angiogenic activity of acidic or basic fibroblast growth factor (FGF) or hepatocyte growth factor (HGF), an antibody capable of inhibiting or neutralizing the coagulant activities of tissue factor, protein C, or protein S, an antibody capable of binding to HER2 receptor, or one or more conventional therapeutic agents such as, for example, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Such other agents may be present in the composition being administered or may be administered separately. Also, the agent is suitably administered serially or in combination with radiological treatments, whether involving irradiation or administration of radioactive substances.

In one embodiment, vascularization of tumors is attacked in combination therapy. The agent of the invention may be administered to tumor-bearing patients at therapeutically effective doses as determined for example by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein, or heat or radiation.

Since the auxiliary agents will vary in their effectiveness, it is desirable to compare their impact on the tumor by matrix screening in conventional fashion. The administration of an agent of the invention, such as an antibody, and TNF is repeated until the desired clinical effect is achieved. Alternatively, the anti-VEGF agent may be administered together with TNF and, optionally, auxiliary agent(s). In instances where solid tumors are found in the limbs or in other locations susceptible to isolation from the general circulation, the therapeutic agents described herein are administered to the isolated tumor or organ. In other embodiments, a FGF or platelet-derived growth factor (PDGF) antagonist, such as an anti-FGF or an anti-PDGF neutralizing antibody, is administered to the patient in conjunction with the agent of the invention. Treatment with an agent of the invention may be suspended during periods of wound healing or desirable neovascularization.

An agent of the invention can be administered alone or in combination with one or more additional therapies such as chemotherapy radiotherapy, immunotherapy, surgical intervention, or any combination of these. Long-term therapy is equally possible as is adjuvant therapy in the context of other treatment strategies.

Antibodies of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering antibodies of the invention that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides methods and compositions for the specific destruction of cells (e.g., the destruction of tumor cells) by administering antibodies of the invention in association with toxins or cytotoxic prodrugs. In specific embodiments, the invention provides compositions and in vitro or in vivo methods for the specific destruction of cells expressing a VEGF-D receptor by contacting VEGF-D receptor-expressing cells with antibodies of the invention in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

In certain embodiments, therapeutic agents of the invention can be used alone. Alternatively, the agents may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject therapeutic agent.

A wide array of conventional compounds has been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplatic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a therapeutic agent of the present invention is administered in combination with another conventional anti-neoplastic agent, either concomitantly or sequentially, such therapeutic agent may be found to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

Pharmaceutical compounds that may be used for combinatory anti-tumor therapy include, merely to illustrate: aminoglutethimide, amsacrine, anastrozole, asparaginase, BCG, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Certain chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (e.g., VEGF inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules”, such as bFGF (basic fibroblast growth factor); (2) neutralizers of angiogenic molecules, such as an anti-bFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. In addition, there are a wide variety of compounds that can be used to inhibit angiogenesis, for example, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naladase inhibitors.

An agent such as an antibody of the present invention may also be administered in combination with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include platelet factor 4; protamine sulphate; sulphated chitin derivatives (prepared from queen crab shells); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cis-hydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3; Chymostatin; Cyclodextrin Tetradecasulfate; Eponemycin; Camptothecin; Fumagillin; Gold Sodium Thiomalate; anticollagenase-serum; alpha2-antiplasmin; Bisantrene; Lobenzarit disodium (N-(2)-carboxyphenyl-4-chloroanthronilic acid disodium or “CCA”; Thalidomide; Angostatic steroid; AGM-1470; carboxynaminolmidazole; and metalloproteinase inhibitors such as BB94.

Administration “in combination with” a further therapeutic agent includes simultaneous (concurrent) and consecutive administration in any order.

It is especially advantageous to formulate the veterinary or pharmaceutical compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Alternatively, the compositions may be presented in multi-dose form.

Examples of dosage units include sealed ampoules and vials and may be stored in a freeze-dried condition requiring only the addition of the sterile liquid carrier immediately prior to use.

The compositions may also be included in a container, pack, or dispenser together with instructions for administration.

Dosages and desired drug concentrations of pharmaceutical or veterinary compositions of the present invention may vary depending on the particular use envisioned. The determination of the appropriate dosage or route of administration is well within the skill of an ordinary physician. In certain embodiments, the antibody is administered at a dose between about 10 ng/kg to 100 mg/kg, 2 μg/kg to 50 mg/kg, 0.1 mg/kg to 30 mg/kg, or 0.1 mg/kg to 10 mg/kg.

When in vivo administration of an agent of the invention is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature. It is anticipated that different compositions will be effective for different treatment compounds and different disorders, that administration targeting one organ or tissue, for example, may necessitate delivery in a manner different from that to another organ or tissue.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and y-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulthydryl residues, lyophilizing from acidic solutions, controlling moisture content, using appropriate additives, and developing specific polymer matrix compositions.

Where sustained-release administration of an agent of the invention is desired in a composition with release characteristics suitable for the treatment of angiogenesis and/or cancer, for example, requiring administration of the agent of the invention, microencapsulation of the agent is contemplated. The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Microencapsulation of recombinant proteins for sustained release has been successfully performed with human growth hormone (rhGH), interferon- (rhIFN-), interleukin-2, and MN rgp120.

The sustained-release compositions of these proteins were developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition.

If the VEGF-D is intracellular and whole antibodies are used to treat a condition, internalizing antibodies are preferred. However, lipofections or liposomes can also be used to deliver the antibody or antibody fragment into cells.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The composition herein may also contain more than one active compound as necessary for the particular indication being treated, preferably those with complementary activities that do not adversely affect each other. Alternatively, or in addition, the composition may comprise an agent that enhances its function. Such molecules are suitably present in combination in amounts that are effective for the purpose intended.

Methods and Uses

The present invention provides methods of inhibiting angiogenesis, particularly dysregulated angiogenesis or angiogenesis in rapidly growing tissues and to inhibit growth of tumors in a subject.

The present invention also includes a method of treating angiogenesis and/or a cancer which is associated with over-expression of VEGF-D and also relates to uses of an agent according to the invention. Alternatively, the invention provides a method of treating a condition responsive to neutralizing VEGF-D. In one embodiment, the condition may include dysregulated angiogenesis, dysregulated lymphangiogenesis, rheumatoid arthritis, psoriasis, lymphangiolieomyomatosis, or other inflammatory condition.

“Treating” or “treatment” refers to both therapeutic treatment and prophylactic or preventative measures, wherein the aim is to prevent, ameliorate, reduce or slow down (lessen) angiogenesis and/or cancer.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition. Successful prevention can be demonstrated in a study involving multiple subjects in which a group that receives a therapeutic agent has either fewer incidences or delayed incidences of the condition, disease, disorder, or phenotype compared to a similar control group that receives only placebo.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The “subject” includes a mammal. The mammal may be a human, or may be a domestic, zoo, or companion animal. While it is particularly contemplated that the methods of the invention are suitable for medical treatment of humans, they are also applicable to veterinary treatment, including treatment of companion animals such as dogs and cats, and domestic animals such as horses, cattle and sheep, or zoo animals such as felids, canids, bovids, and ungulates. A subject may be afflicted with dysregulated angiogenesis and/or cancer or other disorder, or may not be afflicted with dysregulated angiogenesis and/or cancer or other disorder (i.e., free of detectable disease).

The method comprises administering to a subject in need thereof a therapeutically effective amount of a pharmaceutical or veterinary composition adequate to inhibit VEGF-D and/ or comprising an antibody of the present invention that binds specifically to and neutralizes VEGF-D.

The term “therapeutically effective amount” refers to an amount of the agent capable of reducing VEGF-D activity in a subject or mammal to a level which is beneficial to treat angiogenesis and/or cancer or other condition. A therapeutically effective amount may be determined empirically and in a routine manner in relation to treating angiogenesis and/or cancer or other condition, and will result in increased life expectancy.

In yet another embodiment, the invention relates to a method of treating angiogenesis and/or cancer or other condition which comprises, delivering an antibody of the invention via a vector directing expression of a nucleic acid molecule of the invention in vivo in order to treat said subject.

An agent according to the invention may be useful in the treatment of various neoplastic and non-neoplastic diseases and disorders. Thus, the present invention provides a method of treating an angiogenesis-related disease and/or disorder described herein or otherwise known in the art, comprising administering to an individual in need thereof a therapeutically effective amount of an agent of the invention.

Neoplasms and related conditions, cancers, tumors, malignant and metastatic conditions, tissues and organs which can be treated with an agent of the invention include, but are not limited to, abnormal vascular proliferation associated with phakomatoses, advanced malignancies, arrhenoblastomas, astrocytoma, biliary tract, bladder, blood born tumors such as leukemias, brain, breast, cavernous hemangioma, cervix, choriocarcinoma, colon, colorectal, edema (such as that associated with brain tumors), endometriosis, endometrium, esophagus, fibrosarcomas, gastric carcinomas, glioblastoma, head and neck cancer, hemangioblastoma, hemangioma, hepatoblastoma, Kaposi's sarcoma, kidney, larynx, leiomyosarcoma, liver, lung, medulloblastoma, Meigs' syndrome, melanoma, nasopharyngeal carcinoma, neuroblastomas, non-small cell lung cancer, oligodendroglioma, osteogenic sarcoma, ovarian, pancreas, parotid, primary tumors and metastases, prostate, rectum, renal cell, retinoblastoma, rhabdomyosarcoma, Schwannoma, skin, solid tumors, stomach, testes, thecomas, thyroid, urinary tract, uterus, and Wilm's tumor.

In one embodiment, the antibodies may be delivered topically, in order to treat cancers such as skin cancer, head and neck tumors, breast tumors, and Kaposi's sarcoma. In other embodiments, antibodies may be utilized to treat superficial forms of bladder cancer by, for example, intravesical administration.

An agent such as an antibody may be delivered directly into the tumor, or near the tumor site, via injection or a catheter. Of course, as the person skilled in the art will appreciate, the appropriate mode of administration will vary according to the cancer to be treated.

An agent of the invention may be useful in treating other disorders, besides cancers, which involve angiogenesis. Non-neoplastic conditions that are amenable to treatment include acoustic neuromas, age-related macular degeneration, angiofibroma, arteriovenous malformations, artheroscleric plaques, ascites, atherosclerosis, benign tumors, cerebral collaterals, chronic inflammation, corneal graft rejection and other tissue transplantation rejection, coronary collaterals, Crohn's disease, delayed wound healing, diabetic and other proliferative retinopathies, endometriosis, fibromuscular dysplasia, granulations, hemangiomas, hemophiliac joints, hypertrophic scars (keloids), ischemic limb angiogenesis, lung inflammation, macular degeneration, myocardial angiogenesis, neovascular glaucoma, nephrotic syndrome, neurofibromas, nonunion fractures, ocular angiogenic diseases, Osler-Webber Syndrome, pericardial effusion (such as that associated with pericarditis), plaque neovascularization, pleural effusion, preeclampsia, psoriasis, pyogenic granulomas, retinoblastoma, retinopathy of prematurity, retrolental fibroplasia, rheumatoid arthritis, rubeosis, scleroderma, telangiectasia, thyroid hyperplasias (including Grave's disease), trachoma, uvietis and Pterygia (abnormal blood vessel growth) of the eye, vascular adhesions, vasculogenesis, and wound granulation.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the agent of the present invention is expected to be especially useful in reducing the severity of AMD.

In one embodiment of the present invention, methods are provided for treating hypertrophic scars and keloids, comprising the step of administering antibodies of the invention to a hypertrophic scar or keloid. Antibodies of the invention may be injected directly into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development.

Ocular disorders associated with neovascularization which can be treated with an agent of the present invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization.

Thus, one embodiment of the present invention provides a method for treating neovascular diseases of the eye such as corneal neovascularization (including corneal graft neovascularization), comprising the step of administering to a patient a therapeutically effective amount of an agent (including antibodies) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

In one embodiment, an antibody of the invention may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. The anti-angiogenic composition may be prepared with a muco-adhesive polymer which binds to cornea. The anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

The antibodies described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2 to 3 times per year. A steroid could also be added to the injection solution to reduce inflammation resulting from the injection itself.

An agent of the invention may be used for treating neovascular glaucoma, comprising the step of administering to a patient a therapeutically effective amount of an agent such as an antibody to the eye, such that the formation of blood vessels is inhibited. In one embodiment, the agent may be administered topically to the eye in order to treat early forms of neovascular glaucoma. Within other embodiments, the agent may be implanted by injection into the region of the anterior chamber angle. Within other embodiments, the agent may also be placed in any location such that the agent is continuously released into the aqueous humor. In another embodiment of the present invention, methods are provided for treating proliferative diabetic retinopathy, comprising the step of administering to a patient a therapeutically effective amount of a an antibody to the eyes, such that the formation of blood vessels is inhibited.

In one embodiment of the invention, proliferative diabetic retinopathy may be treated by injection of an agent into the aqueous humor or the vitreous, in order to increase the local concentration of the antibodies in the retina. Preferably, this treatment should be initiated prior to the acquisition of severe disease requiring photocoagulation.

In another embodiment of the present invention, methods are provided for treating retrolental fibroplasia, comprising the step of administering to a patient a therapeutically effective amount of an antibody to the eye, such that the formation of blood vessels is inhibited. The compound may be administered topically, via intravitreous injection and/or via intraocular implants.

Moreover, disorders and/or states, which can be treated, prevented, diagnosed, and/or prognosed with an agent such as an antibody of the invention include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one embodiment relating to a method of birth control, an amount of the agent sufficient to block embryo implantation is administered before or after intercourse and fertilization have occurred, thus providing an effective method of birth control, possibly a “morning after” method. Antibodies may also be used in controlling menstruation or administered as either a peritoneal lavage fluid or for peritoneal implantation in the treatment of endometriosis.

Antibodies of the present invention may be incorporated into surgical sutures in order to prevent stitch granulomas.

Antibodies may be utilized in a wide variety of surgical procedures. For example, within one embodiment of the present invention, a composition (in the form of, for example, a spray or film) may be utilized to coat or spray an area prior to removal of a tumor, in order to isolate normal surrounding tissues from malignant tissue, and/or to prevent the spread of disease to surrounding tissues. Within another embodiment of the present invention, compositions (e.g., in the form of a spray) may be delivered via endoscopic procedures in order to coat tumors, or inhibit angiogenesis in a desired locale. Within yet another embodiment of the present invention, surgical meshes which have been coated with a composition comprising an agent of the invention having anti-angiogenic activity, for example an anti-VEGFD antibody, may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

According to further embodiments of the present invention, methods are provided for treating tumor excision sites, comprising administering an agent of the invention such as an antibody to the resection margins of a tumor subsequent to excision, such that the local recurrence of cancer and the formation of new blood vessels at the site is inhibited. Within one embodiment of the invention, the anti-angiogenic agent, for example an antibody, is administered directly to the tumor excision site (e.g., applied by swabbing, brushing or otherwise coating the resection margins of the tumor with the antibody). Alternatively, the antibodies may be incorporated into known surgical pastes prior to administration. Embodiments of the invention contemplate antibodies that may be applied after hepatic resections for malignancy, and after neurosurgical operations.

Within one embodiment of the present invention, antibodies may be administered to the resection margin of a wide variety of tumors, including for example, breast, colon, brain and hepatic tumors. For example, within one embodiment of the invention, an antibody may be administered to the site of a neurological tumor subsequent to excision, such that the formation of new blood vessels at the site is inhibited.

An agent such as an antibody of the invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, agents of the invention can be used as a marker or detector of a particular immune system disease or disorder.

An agent such as an antibody of the invention may be useful in treating or detecting deficiencies or disorders of hematopoietic cells. For example, an antibody could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

An agent such as an antibody can also be used to modulate hemostatic (the stopping of bleeding) or thrombolytic activity (clot formation). For example, by increasing hemostatic or thrombolytic activity, antibodies could be used to treat blood coagulation disorders (e.g., afibrinogenemia, factor deficiencies), blood platelet disorders (e.g. thrombocytopenia), or wounds resulting from trauma, surgery, or other causes. Alternatively, agents that can decrease hemostatic or thrombolytic activity could be used to inhibit or dissolve clotting, important in the treatment of heart attacks (infarction), strokes, or scarring.

An agent such as an antibody may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of an agent that can inhibit an immune response, particularly the proliferation, differentiation, or chemotaxis of T-cells, may be an effective therapy in preventing autoimmune disorders.

Examples of autoimmune disorders that can be treated or detected by an agent such as an antibody of the invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by agent such as an antibody of the invention. Moreover, an agent can be used to treat anaphylaxis, hypersensitivity to an antigenic molecule, or blood group incompatibility.

An agent such as an antibody may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of an agent such as an antibody that inhibits an immune response, particularly the proliferation, differentiation, or chemotaxis of T-cells, may be an effective therapy in preventing organ rejection or GVHD.

Similarly, an agent such as an antibody of the invention may also be used to modulate inflammation. For example, antibodies may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

An agent such as an antibody of the invention can be used to treat or detect hyperproliferative disorders, including neoplasms. An agent may inhibit the proliferation of the disorder through direct or indirect interactions. Alternatively, an agent may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by an agent such as an antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, brain, nervous (central and peripheral), lymphatic system, pelvic, skin, soft tissue, spleen, prostate, thoracic, and urogenital.

In some embodiments, an agent may be used to treat, prevent or ameliorate breast cancer. In other embodiments, an agent may be used to treat, prevent or ameliorate brain cancer. In some embodiments, an agent may be used to treat, prevent or ameliorate head and/or neck cancer. In other embodiments, an agent may be used to treat, prevent or ameliorate prostate cancer. In other preferred embodiments, an agent may be used to treat, prevent or ameliorate colon cancer. In other embodiments, an agent may be used to treat, prevent or ameliorate Kaposi's sarcoma.

Similarly, other hyperproliferative disorders can also be treated or detected by an agent such as an antibody of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The biological activity of an agent of the invention can be measured by standard assays known in the art. Examples include ligand binding assays and Scatchard plot analysis; receptor dimerization assays; cellular phosphorylation assays; tyrosine kinase phosphorylation assays; endothelial cell proliferation assays such as BrdU labeling and cell counting experiments; VEGF-D-dependent cell proliferation assays; and angiogenesis assays. Methods for measuring angiogenesis are standard. Angiogenesis can be assayed by measuring the number of non-branching blood vessel segments (number of segments per unit area), the functional vascular density (total length of perfused blood vessel per unit area), the vessel diameter, the formation of vascular channels, or the vessel volume density (total of calculated blood vessel volume based on length and diameter of each segment per unit area). These assays can be performed using either purified receptor or ligand or both, and can be performed in vitro or in vivo. These assays can also be performed in cells using a genetically introduced or the naturally-occurring ligand or receptor or both. An agent that inhibits the biological activity of VEGF-D will cause a decrease of at least 10%, preferably 20%, 30%, 40%, or 50%, and more preferably 60%, 70%, 80%, 90% or greater decrease in the biological activity of VEGF-D. The inhibition of biological activity can also be measured by the IC₅₀. Preferably, an agent that inhibits the biological activity of VEGF-D will have an IC₅₀ of less than 100 nM, more preferably less than 10 nM and most preferably less than 1 nM.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

All references, including any patents or patent application, cited in this specification are hereby incorporated by reference. No admission is made that any reference constitutes prior art. It will be clearly understood that, although a number of prior art publications are referred to herein, this reference does not constitute an admission that any of these documents form part of the common general knowledge in the art, in Australia or in any other country.

### EXAMPLES

The invention is now further described in detail by reference to the following examples. The examples are provided for purposes of illustration only, and are not intended to be limiting unless otherwise specified. Thus, the invention encompasses any and all variations which become evident as a result of the teaching provided herein.

Recombinant human VEGF-D was obtained from: Source 1, Commonwealth Scientific and Industrial Research Organisation (CSIRO) providing mature dimeric VEGF-D; and Source 2, R&D Systems (Catalog Number 622-VD/CF) providing purified VEGF-D monomer. Full-length human VEGF-D was also obtained as conditioned medium from CHO cells transfected to express human VEGF-D (Source 3).

### Example 1

Several human frameworks were chosen, as scFv fragments, for initial assessment as humanized anti-VEGF-D antibodies. These were referred to as 1A, 3A and 4A. SEQ ID NOs: 1, 3 and 4 comprise the variable frameworks of 1A, 3A and 4A, respectively.

TABLE 1
Summary of framework variations within the
V_{H} domain of scFv frameworks 3A and 4A relative
to scFv framework 1A as defined by SEQ ID NO: 1

| RESIDUE WITHIN | AMINO ACID | FRAMEWORK |
|---|---|---|
| SEQ ID NO: 1 | VARIATION | REGION |
| Q1 | Q1E | FR1 |
| A9 | A9S | FR1 |
| V11 | V11L | FR1 |
| Q13 | Q13K | FR1 |
| A16 | A16E | FR1 |
| V18 | V18L | FR1 |
| V20 | V20I | FR1 |
| A24 | A24G | FR1 |
| T28 | T28S | FR1 |
| A40 | A40M | FR2 |
| Q43 | Q43K | FR2 |
| R67 | R67Q | FR3 |
| V68 | V68F | FR3 |
| T69 | T69V | FR3 |
| M70 | M70F M70I | FR3 |
| T71 | T71S | FR3 |
| T72 | T72A T72L | FR3 |
| T74 | T74K | FR3 |
| T76 | T76V T76I | FR3 |
| M81 | M81L | FR3 |
| E82 | E82Q | FR3 |
| L83 | L83I L83W | FR3 |
| R84 | R84S | FR3 |
| R87 | R87K | FR3 |
| S88 | S88A | FR3 |
| D89 | D89E D89S | FR3 |
| V93 | V93M | FR3 |
| R110 | R110Q | FR4 |
| L113 | L113T | FR4 |
| A117 | A117S | FR4 |

### Example 2

Characterization of scFv Antibodies

Kinetic values were obtained for the three humanized scFv variants, 1A, 3A and 4A (Table 2). All isolated scFv samples were purified as monomer peaks by anti-FLAG affinity and gel filtration chromatography. BIAcore™ analysis was performed by directly immobilizing VEGF-D on a CM5 surface using amine coupling, with the scFv run as the analyte. Analysis of binding was carried out using the BIAevaluation kinetic analysis software. All repeats were separate protein expressions and were performed at 25° C.

TABLE 2
Overview of the humanized scFv variants

| Clone | Kₐ (1/Ms) | K_{d} (1/s) | K_{D} (nM) |
|---|---|---|---|
| 1A* | 7.69 × 10⁵ | 1.09 × 10⁻² | 13.1 |
| 3A | 4.23 × 10⁵ | 2.15 × 10⁻³ | 5.1 (scFv dimer) |
| 4A | 5.25 × 10⁵ | 7.88 × 10⁻³ | 15.0 |

*Average of several repeats, see Table 3.
scFv 1A was selected for further analysis and potential optimization.

Expression in E. coli and Purification of the 1A scFv and Other scFv Variants

Small Scale Expression

ScFvs were expressed on a small scale (10 ml) in E. coli using standard methodology and assessed for expression into the supernatant. In all cases, expression levels were good and detectable levels of scFv were present in the supernatant.

The small scale scFv expressions were used to prepare crude periplasmic fractions that were assessed for binding activity against VEGF-D using a BIAcore™ biosensor. The ability of the scFv to bind VEGF-D in these BIAcore™ assays indicated that they tended to fold adequately in E. coli, forming functional binding proteins.

Large Scale Expression and Purification

To establish the base levels of binding between 1 A scFv and VEGF-D, larger scale E. coli expressions were conducted. There were no issues with the expression of 1A scFv. Purification and concentration of the 1A scFv preparations generated sufficient amounts of purified protein for subsequent analysis and did not result in any significant aggregation.

Establishing a Baseline Affinity for 1A scFv

The binding of purified 1A scFv to VEGF-D was analyzed using a BIAcore™ biosensor (Table 3). Overall, the data indicated that 1A scFv bound to VEGF-D with an affinity of around 13.1 nM.

TABLE 3
Complete Analysis of Binding Data for 1A scFv

| | Kₐ (1/Ms) | K_{d} (1/s) | K_{D} (nM) |
|---|---|---|---|
| Repeat 1 | 7.69 × 10⁵ | 1.09 × 10⁻² | 14.2 |
| Repeat 2 | 1.06 × 10⁶ | 1.10 × 10⁻² | 10.4 |
| Repeat 3 | 7.07 × 10⁵ | 1.04 × 10⁻² | 14.8 |
| Average | 8.45 × 10⁵ | 1.08 × 10⁻² | 13.1 |

BIAcore™ Off-Rate

Screening based on scFv binding properties involved surface plasmon resonance (SPR) measurements with a BIAcore™ biosensor. scFv variants were expressed in 200 μL E. coli cultures and the supernatants used to generate crude off-rate only kinetics, which were assessed “relative” to 1A, or other variants.

Full BIAcore™ Characterization of scFv Variants

scFv variants were fully characterized using a BIAcore™ biosensor. All isolated scFv samples were purified as monomer peaks by anti-FLAG affinity chromatography followed by gel filtration chromatography. BIAcore™ analysis was performed by directly immobilizing VEGF-D on a CM5 surface using amine coupling, with the scFv run as the analyte. Analysis of binding was carried out using the BIAevaluation kinetic analysis software. All repeats were separate protein expressions and were performed at 25° C.

svFv Variants

Variants of 1A scFv were constructed from the existing expression plasmid by introducing point mutations into the RNA. Approximately 1-2 nucleotide changes were made every 750 base pairs. This resulted in a panel of scFv variants containing from 1 to 4 amino acid modifications. A number of rounds of mutagensis were undertaken. In total, 19,840 clones were screened using ELISA protocols (not described).

Results

The ELISA screens identified a combined total of 586 variants with apparent improved affinity for VEGF-D relative to the 1A. These 586 variants were analyzed by BIAcore™ to establish their off-rate kinetics. These more detailed analysis identified 21 unique scFv variants that showed improvements in binding to VEGF-D (particularly with regards to the off-rate). The final pool of 21 scFv clones also exhibited good expression characteristics in E. coli.

Table 4 shows the unique amino acid variations identified from the clones that were selected from the ELISA and SPR screens.

TABLE 4
Off-Rate Ranking of scFv Clones

| | Heavy Chain | Light Chain | |
|---|---|---|---|
| Clone | Mutations* | Mutations* | kd (1/s) |
| A63 | E10K, V20A | | 3.27 × 10⁻³ |
| F61 | E82K | | 3.35 × 10⁻³ |
| D69 | Y95H | D198G | 3.41 × 10⁻³ |
| K89 | S25R | | 4.03 × 10⁻³ |
| W25 | E46G | | 4.60 × 10⁻³ |
| G11 | | D198G | 4.64 × 10⁻³ |
| Z49 | D89G | | 4.97 × 10⁻³ |
| Z23 | E46G, G131R** | | 5.56 × 10⁻³ |
| G37 | Q3R, S100G^{H3} | | 5.86 × 10⁻³ |
| W36 | E46G | | 6.11 × 103 |
| AC75 | M81V | | 6.37 × 10⁻³ |
| H71 | K19R, T69A | S158T^{L1} | 6.53 × 10⁻³ |
| H44 | E46G, G131R** | V221M | 6.57 × 10⁻³ |
| I71 | K19R | | 6.61 × 10⁻³ |
| AA09 | T241A | | 6.95 × 10⁻³ |
| T07 | T208A | | 7.01 × 10⁻³ |
| G75 | A40V, T112A | | 7.20 × 10⁻³ |
| Q30 | T112A | | 7.20 × 10⁻³ |
| I84 | A40V | | 7.38 × 10⁻³ |
| G38 | A40V, V114A | | 7.72 × 10⁻³ |
| P88 | | T138I | 7.85 × 10⁻³ |
| V37 | A40V | | 7.94 × 10⁻³ |
| AA16 | V2A | | 7.95 × 10⁻³ |
| AR74 | D73G; E82K; T99A^{H3} | | 1.67 × 10⁻³ |
| AW61 | V68A; E82K | | 2.22 × 10⁻³ |
| CJ17 | K23E; E82K | | 2.91 × 10⁻³ |
| DI26 | E10K; V20A; N55S^{H2} | | 3.61 × 10⁻³ |
| BF36 | K63R; E82K; T112A | K212R | 3.62 × 10⁻³ |
| DD25 | E10K; V20A; M48V | | 3.73 × 10⁻³ |
| CI67 | V68A; E82K | T230A^{L3} | 3.79 × 10⁻³ |
| DA18 | E10K; V20A | F193S^{L2} | 4.01 × 10⁻³ |
| DK53 | E10K; V20A | D197G | 4.06 × 10⁻³ |
| CZ77 | E10K; V20A; K65R^{H1} | | 4.08 × 10⁻³ |
| DA74 | V5A; E10K; V20A | F193S^{L2} | 4.24 × 10⁻³ |
| CK61 | E10K; E82K | | 5.22 × 10⁻³ |
| DB29 | E10K; V20A | S144G | 5.75 × 10⁻³ |
| DD76 | E10K; V20A; S75N | | 6.37 × 10⁻³ |
| DC56 | E10K; K12R; V20A | | 7.23 × 10-³ |
| CW16 | D89G | V146A | 7.26 × 10⁻³ |
| CJ37 | E82K | Q238R | 7.32 × 10⁻³ |
| DC64 | E10K; V18M; V20A | | 7.38 × 10⁻³ |
| CX03 | K19T | D198G | 7.39 × 10⁻³ |
| CZ24 | E10K; S17N; V20A | | 7.59 × 10⁻³ |
| DE03 | E10K; V20A; S25G | | 7.98 × 10⁻³ |
| CZ13 | V5A; E10K; V20A | | 8.26 × 10⁻³ |
| DD17 | E10K; S17G; V20A | | 9.10 × 10⁻³ |
| HH41 | E82K | S165G^{L1} | 1.04 × 10⁻³ |
| HU75 | E82K | S144G; Q238R | 1.81 × 10⁻³ |
| HW78 | S25R; S77G; E82K | | 1.89 × 10⁻³ |
| HH69 | E10K; V20A; N55S^{H2} | S144G; Q238R | 2.12 × 10⁻³ |
| HX28 | E10K; V20A; N55S^{H2} | D198G | 6.43 × 10⁻³ |

Numbering is derived from SEQ ID NO: 25.
*Variations that occur within a CDR are marked in superscript:
^{H1}heavy chain CDR1;
^{H2}heavy chain CDR2;
^{H3}heavy chain CDR3;
^{L1}light chain CDR1;
^{L2}light chain CDR2;
^{L3}light chain CDR3.
**Mutation occurs in scFv linker region.

Variation Analysis

Variations were spread throughout the heavy and light chain with 4 distinct variant hot spots (i.e. 5 or more variations at the same position isolated from independent clones) at amino acids 10 (E10K), 20 (V20A), 55 (N55S) and 82 (E82K). One hot spot occurred within a CDR region (N55S); three hot spots were located within FRs of the heavy chain. Multiple variants also carried light chain variations at amino acids 144 (S144G) and 198 (D198G). The variation diversity recovered in the heavy chain was greater than that seen in the light chain. Many of the variants were recovered in various combinations with each other. For example, the E82K variant (originally recovered in clone F61) and the E10K variant (recovered in clone A63) were isolated in combination in clone CK61. Similarly, the D198G variant and the E10K+V20A+N55S variants (recovered in clone DI26) were isolated in combination in clone HX28.

Although the final spectrum of variations isolated with each clone improves the overall binding of the scFv, the contribution or “weight” of individual variations is often difficult to predict and there are often unpredictable cumulative effects. While not wishing to be bound to any particular theory, it appears that CDR variations at hotspots such as N55S most likely contribute to binding by directly influencing the interaction of the scFvs with the targeted epitope on VEGF-D. Framework changes such as the heavy chain variant E82K or the light chain variant S144G probably have a more subtle effect on binding, acting to shift the CDRs relative to the target epitope, or to reposition the heavy and light chains with respect to each other. The Q238R variant did not appear to influence binding affinity to any degree and is possibly a scFv stabilizing, folding or expression change.

Final Ranking and Characterization of scFv Variants

Clones F61, A63, AR7, AW61, CJ37, DI126 and DA18 were produced in large-scale expression cultures and purified by anti-FLAG affinity chromatography and gel filtration. The binding kinetics against VEGF-D were then measured using a BIAcore™ biosensor as described previously. All seven scFvs (Table 5) showed at least 2.5-fold improvement over scFv 1A, with clones AR74 (2.12 nM), DI26 (3.12 nM) and F61 (3.31 nM) showing a 6.2-fold, 4.2-fold and 4-fold improvement over clone 1A, respectively.

TABLE 5
Characterization of selected scFv variants

| | Heavy Chain | Light Chain | Ka | Kd | K_{D} | |
|---|---|---|---|---|---|---|
| Clone | Mutations* | Mutations* | (1/Ms, ×10⁶) | (1/s, ×10⁻³) | (nM) | Fold^{§} |
| 1A | — | — | 8.45 × 10⁵ | 1.08 × 10² | 13.1 | — |
| F61 | E82K | — | 2.72 | 8.99 | 3.31 | 4.0 |
| A63 | E10K; V20A | — | 1.49 | 7.20 | 4.83 | 2.7 |
| AR74 | D73G; E82K; T99A^{H3} | — | 1.79 | 3.79 | 2.12 | 6.2 |
| AW61 | V68A; E82K | — | 1.96 | 8.28 | 4.23 | 3.1 |
| CJ37 | E82K | Q238R | 2.81 | 9.95 | 3.55 | 3.7 |
| DI26 | E10K; V20A; N55S^{H2} | F193S^{L2} | 2.98 | 9.30 | 3.12 | 4.2 |
| DA18 | E10K; V20A | N191S^{L2} | 2.13 | 10.6 | 4.97 | 2.6 |

^{§}Fold-improvement with respect to clone 1A.

### Example 3

### Characterization of IgG Antibodies

The “wild-type” humanized scFv 1 A and selected scFv variant clones were reformatted into IgGs (e.g. Ig-1A) for further analysis and to confirm that the scFv fragments retained affinity as reformatted IgGs.

Clone 1 A and selected scFv variant clones were subcloned into IgG expression vectors, sequence confirmed and transfected into mammalian cells for IgG production. The IgGs were purified from cell culture supernatants by Protein A affinity chromatography followed by gel filtration, then diluted appropriately for assessment of binding kinetics using a BIAcore™ biosensor.

The biosensor protocols described below were used for various analyses, depending on the specific antibody and species of VEGF-D studied.

The binding of Ig-1A was initially determined to be at 16.1 nM (Table 6). These initial binding experiments established that the scFv 1A retained its binding when reformatted into an IgG (Ig-1A).

TABLE 6
VEGF-D Binding Data for Murine anti-
VEGF-D Monoclonal Antibody and Ig-1A

| Antibody | VEGF-D | ka (1/Ms) | kd (1/s) | K_{D} (nM) | |
|---|---|---|---|---|---|
| Murine IgG | Source 1 | 5.90 × 10⁵ | 1.49 × 10⁻³ | 3.0 | (Protocol A) |
| | | | | 4.6 | (Protocol B) |
| Murine IgG | Source 2 | 0.86 × 10⁵ | 3.27 × 10⁻³ | 38.1 | |
| Ig-1A | Source 1 | 6.80 × 10⁵ | 0.59 × 10⁻³ | 1.8 | (Protocol A) |
| | | | | 0.70 | (Protocol B) |

| | | | | |
|---|---|---|---|---|
| Ig-1A | Source 2 | 4.94 × 10⁵ | 9.01 × 10⁻³ | 16.1 |

*Note that initial characterization of the murine anti-VEGF-D monoclonal antibody was conducted using an alternative capture method.

Direct Murine Antibody Capture

A mouse anti-VEGF-D monoclonal antibody was analyzed using a modification of the protocol described by Achen et al. (Eur. J. Biochem. (2000) 267: 2505-2515), except that 1000 RU of the antibody was immobilized on a CM5 chip surface, compared to 4700 RU immobilized in the initial study. This change was made since the higher levels of monoclonal antibody immobilized in the original protocol would cause significant mass transport limitations and significantly under represent the on-rate value and considerably exaggerate the off-rate value.

Anti-Mouse IgG (Fc) Capture

This protocol was used to measure the binding kinetics of the murine anti-VEGF-D monoclonal antibody against the VEGF-D dimer.

Immobilization of Anti-Mouse Fc Specific IgG

Two flow cells on a CM5 chip were activated for 7 min at 25° C. using a mixture of NHS.EDC. Anti-mouse IgG antibody (BIAcore™, BR-1008-38) was diluted to 30 μg/ml in 10 mM sodium acetate pH 5 and injected over two surfaces for 7-10 min to achieve capture molecule densities of around 10 000 RU. Surfaces were blocked for 7 min with ethanolamine (1M, pH 8.2) and pre-conditioned using five 180 s injections of 10 mM Glycine-HCL pH 1.7. Immobilization and conditioning steps were carried out at a flow rate of 10 μl/min using HBS-N running buffer.

Mouse IgG Characterization

Murine antibody binding experiments were performed at 25° C. using HBS-EP+ running buffer. Analytes were diluted in running buffer and injected for 100-200 s at a flow rate of 10 μl/min to a capture density of 500 RU. The captured surface was allowed to stabilize for 7 min before injection of VEGF-D dimer analyte for 180 s at a flow rate of 70 μl/min and dissociation monitored for 10 min. The antigen surfaces were regenerated with a 3 min injection of 10 mM Glycine-HCL pH 1.7 at a flow rate of 10 μl/min. Data was processed using the reference surface and double referencing from blank injections. Binding constants for the IgG/antigen interactions were determined using data globally fitted to a 1:1 interaction model. Rₘₐₓ parameter was set locally to account for slightly varying amounts of ligand capture. Refractive index was constant (RI=0).

Anti-Human IgG (Fc) Capture

This protocol was used to measure the IgG binding kinetics against VEGF-D.

Immobilization of Anti-Human Fc Specific IgG

Two flow cells on a CM5 chip were activated for 7 min at 25° C. using a mixture of NHS.EDC. Goat anti-human-Fc purified IgG antibody (2mg/ml, Invitrogen) was diluted 1 in 5 in 10 mM sodium acetate pH 5 and injected over two surfaces for 10-15 min to achieve capture molecule densities of around 10 000 RU. Surfaces were blocked for 7 min with ethanolamine (1M, pH 8.2) and pre-conditioned using five 12 s injections of 100 mM H₃PO₄. Immobilization steps were carried out at a flow rate of 10 μl/min using HBS-N running buffer.

IgG Characterization

All IgG binding experiments were performed at 25° C. using HBS-EP+ running buffer. Analytes (Ig-1A and variants) were diluted in running buffer and injected for 100-200 s at a flow rate of 10 μl/min to a capture density of 500 RU. The captured surface was allowed to stabilize for 7 min before injection of VEGF-D for 180s at a flow rate of 70 μl/min and dissociation monitored for 7 min. The antigen surfaces were regenerated with a 12 s pulse of 100mM H₃PO₄. Data was processed using the reference surface and double referencing from blank injections. Binding constants for the IgG/antigen interactions were determined using data globally fitted to a 1:1 interaction model. Rₘₐₓ parameter was set locally to account for slightly varying amounts of ligand capture. Refractive index was constant (RI=0).

Measurement of IgG Binding Against the VEGF-D Dimer

Two different biosensor assay formats were used to measure IgG binding kinetics against the Source 1 VEGF-D dimer.

Protocol A: The first assay format, Protocol A, used an assay system where IgGs were captured on a biosensor chip surface using an anti-human IgG (Fc) antibody and the Source 1 VEGF-D dimer run over the chip as the analyte in solution. This ensured that all IgG molecules were presented to the antigen in an active orientation.

The SPR protocol was slightly modified to compensate for the dimeric nature of the VEGF-D. Ig-1A or IgG variants were analyzed using an anti-human capture kit as described for Anti-human IgG (Fc) capture above, except that 1000 RU of the IgG or IgG variant was captured on the anti-human chip surface. Source 1 VEGF-D was run as the analyte at concentrations of 25 nM, 12.5 nM, 6.25 nM, 3.13 nM, and 1.56 nM. The kinetics generated using this approach are accurate within the experimental parameters set. This standard assay protocol is best suited to studying 1:1 interactions and thus, may not be ideal for analyzing binding between the IgG variants and the VEGF-D dimer. In light of this, Protocol B was also used.

Protocol B: In the second assay format, Protocol B, the orientation of the IgGs and the VEGF-D antigen was reversed relative to Protocol A. The Source 1 VEGF-D dimer was immobilized directly on the chip surface by amine coupling and the murine anti-VEGF-D monoclonal antibody, Ig-1A or IgG variants flowed over this surface as analytes at concentrations of 4 nM, 1.33 nM, and 0.44 nM. This protocol allowed avidity effects to be controlled by reducing the density of dimeric VEGF-D immobilized on the chip and in effect reduce the bivalent binding properties of the system being analyzed.

Antigen Immobilization

All four flow cells on a CM5 chip were activated for 7 min at 25° C. using a mixture of NHS.EDC. Flow cell one was used as an unmodified (activated and blocked) reference against flow cells 2-4. Source 1 VEGF-D dimer was diluted to 1-3 μg/ml into 10 mM sodium acetate, pH 4, and injected over three surfaces for different lengths of time to achieve low ligand densities of 10, 15 and 20 RU to minimize avidity effects of the IgG. All surfaces were blocked for 7 min with ethanolamine (1M, pH 8.2) and pre-conditioned using five 10 s injections of 100 mM H₃PO₄; 1M NaCl. Immobilization steps were carried out at a flow rate of 30 μl/min using HBS-N running buffer.

IgG Characterization

All IgG binding experiments were performed at 25° C. using HBS-EP+ running buffer. Analytes (murine reference anti-VEGF-D monoclonal antibody and humanized variants) were injected for 200 s at a flow rate of 70 μl/min and dissociation monitored for 250 s. The antigen surfaces were regenerated with a 10 s pulse of 100 mM H₃PO₄; 1M NaCl. Analyte concentrations used for murine and humanized variants were 4 nM, 1.33 nM, and 0.44 nM. Data was processed using the reference surface and double referencing from blank injections. Binding constants for the IgG/antigen interactions were determined from the three antigen surfaces globally fitted to a 1:1 interaction model. Refractive index parameter was constant (RI=0).

Binding Results Against the Source 1 VEGF-D Dimer

The interaction between the murine anti-VEGF-D monoclonal antibody and the purified Source 1 VEGF-D dimer was conducted using the anti-mouse IgG (Fc) capture method described above. Binding against the Source 1 VEGF-D dimer was measured using Protocols A and B described above.

In summary, affinities in the picomolar range were observed for the six IgGs listed below using both assay formats (Table 7A and Table 7B). HU75, for example, demonstrated dimer-binding affinities of 127 pM and 400 pM using Protocol A and B, respectively, which equates to an improvement of 23.6-fold or 11.5-fold, respectively, over the original murine anti-VEGF-D monoclonal antibody, and an improvement of 5.5-fold and 4.4-fold, respectively, over humanized Ig-1A. Other variants including HH69, HW78, HH41 and HX28 exhibited improvements of 5.5- to 1.8-fold using Protocol A, and improvements of 5.5- to 2.0-fold using Protocol B, over humanized Ig-1A. Variant DI26 demonstrated improvement over humanized Ig-1A of between 1.0-fold (Protocol A) and 2.0-fold (Protocol B).

TABLE 7A
IgG characterization against Source 1 VEGF-D dimer. Protocol A - IgGs
captured on biosensor chip, VEGF-D dimer run as analyte in solution

| | | | Maintain | | | | |
|---|---|---|---|---|---|---|---|
| | Heavy Chain | Light Chain | Epitope | ka | kd | K_{D} | |
| Clone | Mutations* | Mutations* | Specificity | (1/Ms, ×10⁵) | (1/s, ×10⁻⁵) | (pM) | Fold^{§} |
| Ig-1A | — | — | NA | 7.1 | 49 | 696 | — |
| HU75 | E82K | S144G; Q238R | Yes | 4.6 | 5.8 | 127 | 5.5 |
| HH69 | E10K; V20A; N55S^{H2} | S144G; Q238R | Yes | 3.9 | 7.7 | 201 | 3.5 |
| HW78 | S25R; S77G; E82K | — | Yes | 5.1 | 1.8 | 342 | 2.0 |
| HH41 | E82K | S165G^{L1} | Yes | 5.3 | 19 | 364 | 1.9 |
| HX28 | E10K; V20A; N55S^{H2} | D198G | Yes | 3.6 | 14 | 383 | 1.8 |
| DI26 | E10K; V20A; N55S^{H2} | — | Yes | 5.6 | 39 | 699 | 1.0 |

TABLE 7B
IgG characterization against Source 1 VEGF-D dimer. Protocol B - VEGF-D
immobilized on biosensor chip, IgGs run as analyte in solution

| | | | Maintain | | | | |
|---|---|---|---|---|---|---|---|
| | Heavy Chain | Light Chain | Epitope | ka | kd | K_{D} | |
| Clone | Mutations* | Mutations* | Specificity | (1/Ms, ×10⁵) | (1/s, ×10⁻³) | (pM) | Fold^{§} |
| Ig-1A | — | — | NA | 7.2 | 1.3 | 1800 | — |
| HU75 | E82K | S144G; Q238R | Yes | 48 | 1.9 | 400 | 4.4 |
| HH69 | E10K; V20A; N55S^{H2} | S144G; Q238R | Yes | 30 | 1.0 | 330 | 5.3 |
| HW78 | S25R; S77G; E82K | — | Yes | 54 | 1.8 | 320 | 5.5 |
| HH41 | E82K | S165G^{L1} | Yes | 47 | 1.7 | 360 | 4.9 |
| HX28 | E10K; V20A; N55S^{H2} | D198G | Yes | ND | ND | ND | ND |
| DI26 | E10K; V20A; N55S^{H2} | — | Yes | 13 | 1.2 | 880 | 2.0 |

*Variants that occur within a CDR are marked in superscript.
^{H2}heavy chain CDR2;
^{L1}light chain CDR1.
^{§}Fold-improvement with respect to Ig-1A.

Biosensor analysis of the interaction between the murine anti-VEGF-D monoclonal antibody and Source 2 VEGF-D was conducted using the direct murine antibody capture method described above. Kinetic measurement of Ig-1A against Source 2 VEGF-D was conducted using the anti-human IgG (Fc) capture protocol described above. Affinities in the nanomolar range were observed for the four IgGs listed below (Table 8)

TABLE 8
IgG binding against Source 2 VEGF-D Monomer

| | | | Maintain | | | | |
|---|---|---|---|---|---|---|---|
| | Heavy Chain | Light Chain | Epitope | ka | kd | K_{D} | |
| Clone | Mutations* | Mutations* | Specificity | (1/Ms) | (1/s) | (nM) | Fold^{§} |
| Ig-1A | — | — | NA | 7.3 × 10⁵ | 6.78 × 10⁻³ | 9.2 | — |
| A63 | E10K; V20A | — | Yes | 0.97 | 6.77 × 10⁻³ | 7.0 | 1.3 |
| F61 | E82K | — | Yes | 1.64 | 8.24 × 10⁻³ | 5.0 | 1.8 |
| DI26 | E10K; V20A; N55S^{H2} | — | Yes | 1.0 × 10⁵ | 4.10 × 10⁻³ | 4.1 | 2.2 |
| AW61 | V68A; E82K | — | Yes | 1.76 | 7.25 × 10⁻³ | 4.1 | 2.2 |

*Variants that occur within a CDR are marked in superscript,
^{H2}heavy chain CDR2.
^{§}Fold-improvement with respect to Ig-1A.

### Example 4

A summary of all amino acid substitutions identified in V_{H} domain variants detected in antibodies of mutagenesis-derived clones is provided in Table 9.

TABLE 9
Summary of V_{H} Variants in Specific Clones After Mutagenesis

| | CLONE | | | | | | | | |
|---|---|---|---|---|---|---|---|---|---|
| | F61 | A63 | AW61 | DI26 | HU75 | HH69 | HW78 | HH41 | HX28 |
| DOMAIN | VARIANT | | | | | | | | |
| FR1 | | E10K | | E10K | | E10K | | | E10K |
| FR1 | | V20A | | V20A | | V20A | | | V20A |
| FR1 | | | | | | | S25R | | |
| CDR2 | | | | N55S | | N55S | | | N55S |
| FR3 | | | V68A | | | | | | |
| FR3 | | | | | | | S77G | | |
| FR3 | E82K | | E82K | | E82K | | E82K | E82K | |

FR, framework region;
CDR, complementarity determining region.

### Example 5

A summary of all amino acid substitutions identified in V_{L} domain variants detected in antibodies of mutagenesis-derived clones is provided in Table 10.

TABLE 10
Summary of V_{L} Variants in Specific Clones After Mutagenesis

| CLONE | | | | | | | | |
|---|---|---|---|---|---|---|---|---|
| F61 | A63 | AW61 | DI26 | HU75 | HH69 | HW78 | HH41 | HX28 |

| | |
|---|---|
| DOMAIN | VARIANT |

| | | | | |
|---|---|---|---|---|
| FR1 | S144G | S144G | | |
| CDR1 | | | S165G | |
| FR3 | | | | D198G |
| FR4 | Q238R | Q238R | | |

FR, framework region;
CDR, complementarity determining region.

### Example 6

Testing Neutralizing Activity of IgG Antibodies in VEGFR-2-Ba/F3 ³H-Thymidine Incorporation Bioassay

This assay is broadly described in Achen et al. (Eur. J. Biochem. (2000) 267: 2505-2515).

Background

The VEGFR-2-Ba/F3 cell line expresses the chimeric receptor consisting of the extracellular domain of VEGFR-2 and the transmembrane and cytoplasmic domains of EpoR. Ba/F3 cells are IL-3 dependent. In the absence of IL-3, the VEGFR-2-Ba/F3 cells survive and proliferate only in the presence of growth factors capable of binding and cross-linking the extracellular domain of mouse VEGFR-2.

Methodology

Cell Culture

The VEGFR-2-Ba/F3 cell line was cultured in:
DMEM (Gibco)
10% FBS (Gibco)
10% WEHI-3BD-conditioned medium (source of IL-3)
1 mg/mL G418 (Geneticin, Gibco, stored at 4° C. in dark)
4 mM Glutamax (Gibco)

For the Bioassay, the IL-3 source (i.e. 10% WEHI-3BD-conditioned medium) was omitted.

Cells were passaged by dilution 1:15 every 3 days.

Bioassay Plates

washes were carried out at a spin speed equating to 290 g for 5 min;

the mid-log cells were removed from the medium containing IL-3 and washed 3 times in ice-cold MT-PBS;

the removed cells were washed once in medium lacking IL-3 (i.e. DMEM, 10% FBS, 1 mg/mL G418 and 4 mM Glutamax);

cells were resuspended in approximately 450 μL medium lacking IL-3 at a concentration of 7.4×10⁴ cells/ml (cells counted using 1:1 trypan blue at 0.04% in PBS);

the test sample was added to the cell volume at 10% v/v, i.e. 135 μL cells+15 μL test sample (150 μL/well total volume);

each test sample was tested in triplicate;

135 μL of cells (at 7.4×10⁴ cells/mL) was added to each well (using multi-channel pipette where possible)—this equated to approx 10,000 cells per well.

Growth Factor Titration Curves

15 μL of VEGF-D test sample was added to each well containing 135 μL of VEGFR-2-Ba/F3 cells suspended in IL-3-deficient medium as described above;

VEGF-D samples were serially diluted at a dilution factor of 10 (performed in triplicate);

the samples were prepared so that the final concentration of VEGF-D was 4000, 2000, 1000, 500, 250, 125, 62.5, 31.25, 15.625, 7.8, 3.9 and 0 ng/mL;

wells of VEGFR-2-Ba/F3 cells in 150 μL of IL-3 containing medium were used as the control;

The Effective Concentration (EC) of the VEGF-D for the VEGFR-2-Ba/F3 cells was calculated. Typically, an EC in the range from EC₅₀-EC₈₀ was selected for use in the assay. Based on the EC₅₀-EC₈₀ value, Source 1 VEGF-D (CSIRO) was used at a concentration of 5 ng/mL and Source 2 VEGF-D (R&D Systems) was used at a concentration of 100 ng/mL.

Dose Response of Cells to VEGF-D with Varying Concentrations of Antibody

7.5 μL of antibody was added to each well in a 1:2 serial dilution (in triplicate) to produce concentrations in the wells of 50, 25, 12.5, 6.25, 3.125, 1.5625, 0.78125, 0.39 and 0 (antibody buffer only) μg/mL 7 5 μL of VEGF-D at a concentration of 5 ng/mL (Source 1) and 100 ng/ml (Source 2) was added to each well. The antibody plus VEGF-D mixture was incubated for 1 hour at 4° C. in PBS prior to being added to the cells as described below.

Assay

15 μL of antibody plus VEGF-D, having been diluted and incubated for 1 hour as described above, was added to each well of cells;

The assay plate was incubated for 48 hours at 37° C., 10% CO₂;

50 μL of ³H-thymidine at a concentration of 20 μCi/mL was added to each well (equating to 1 μCi/well);

The assay plates were incubated for 4 hours at 37° C.;

The cells were harvested using a 96-well plate cell harvester and counted using a Liquid Scintillation Counter;

The results were plotted as ³H-thymidine incorporated×10³ (cpm) vs Sample Concentration (ng/mL)

In this example, antibodies F61, HW78, HH69, HH41, HX28, HU75, DI26, and AW61 were tested for neutralizing activity with respect to VEGF-D and VEGF-C. The experiment was conducted twice: Experiment 1 and Experiment 2.

Results

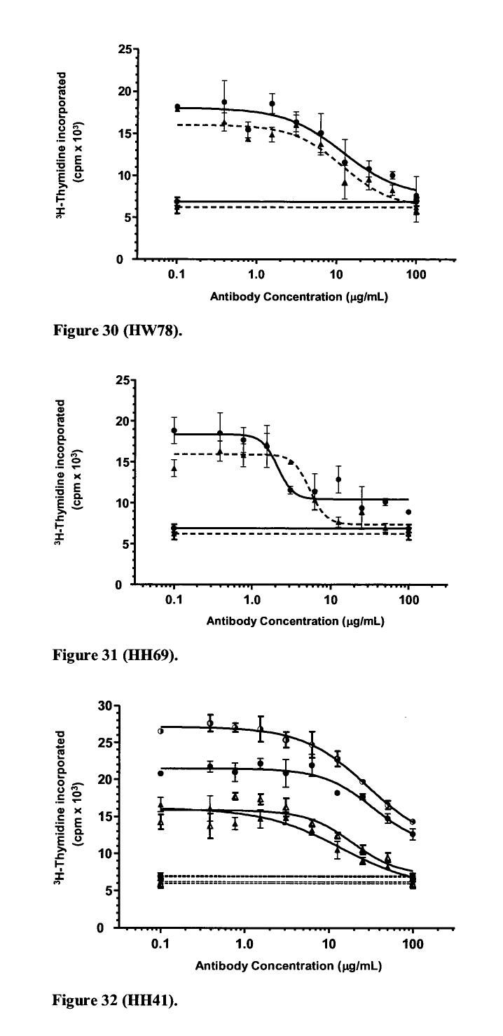

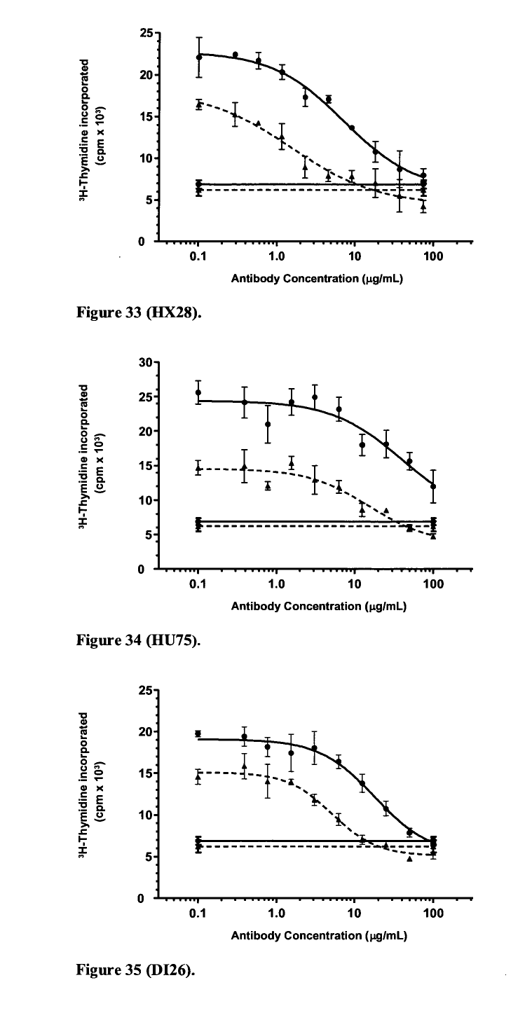

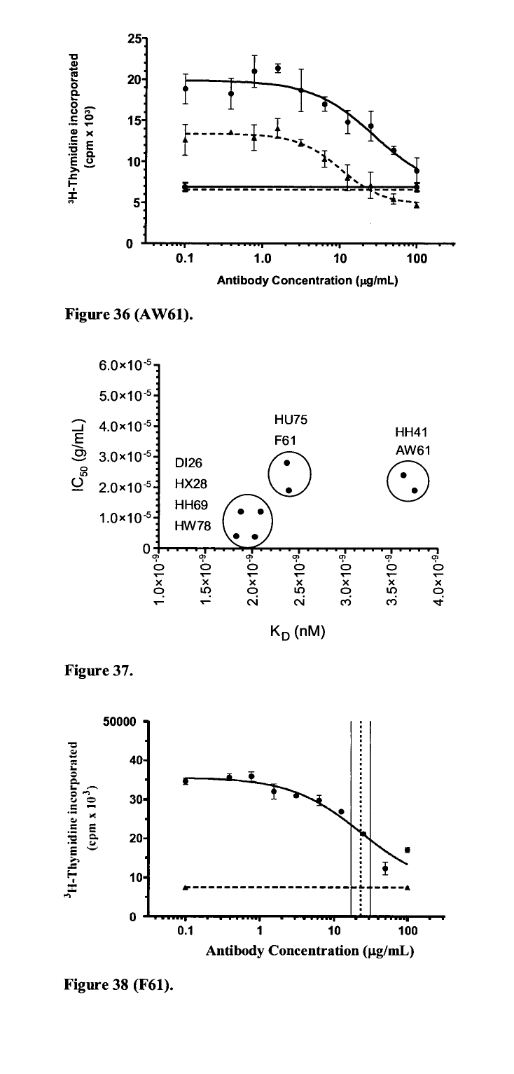

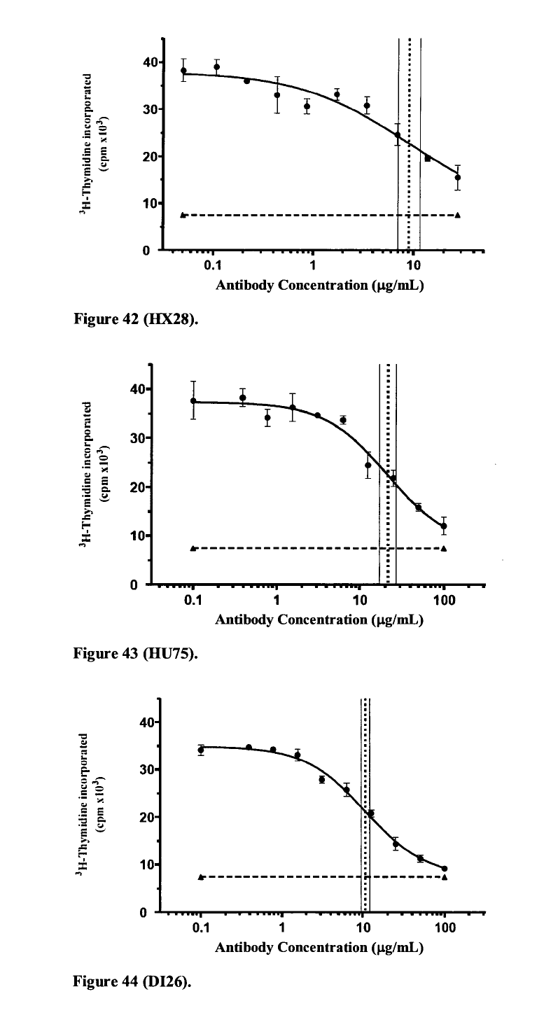

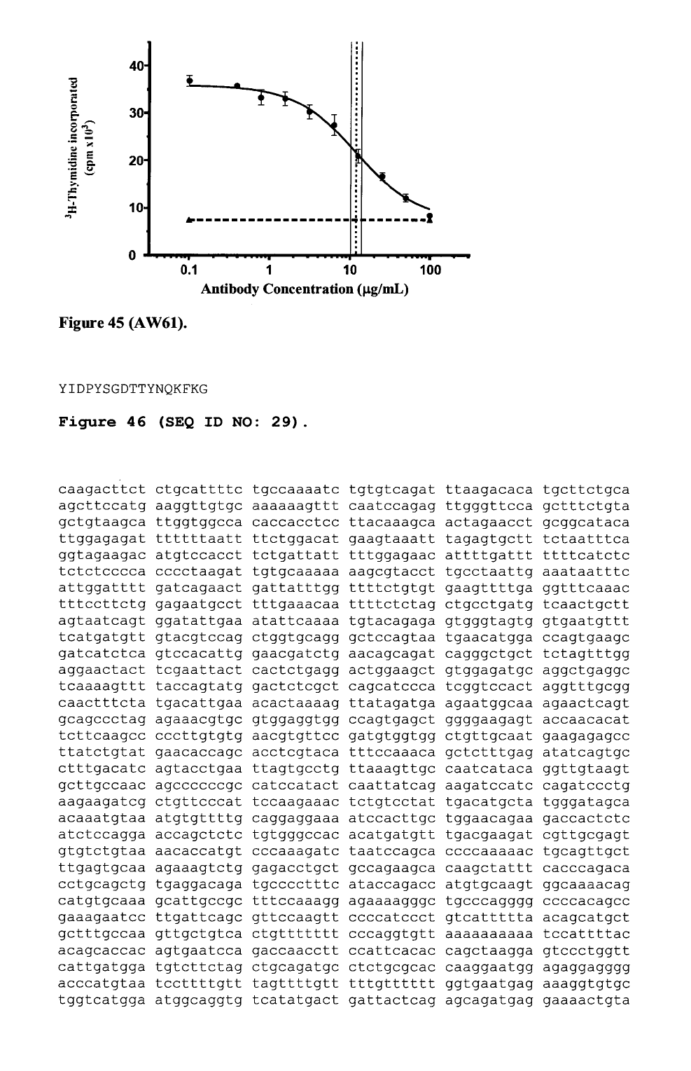

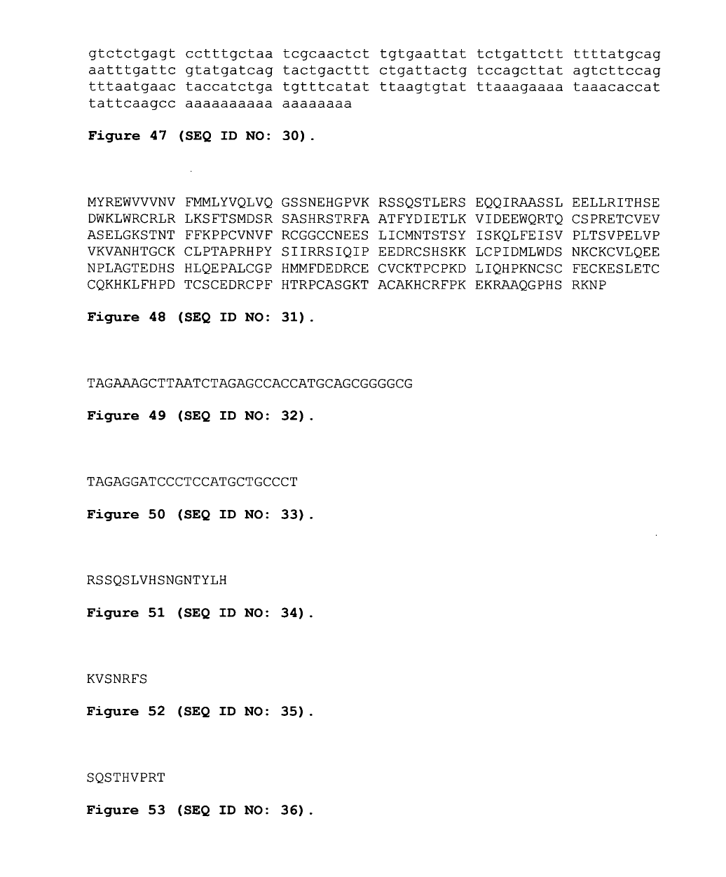

The results of the VEGF-D neutralizing activity of IgG antibodies clones F61, HW78, HH69, HH41, HX28, HU75, DI26, and AW61 of Experiment 1 are presented in FIGS. 29 to 36 and of Experiment 2 are presented in FIGS. 38 to 45. Each figure illustrates inhibition of ³H-thymidine incorporation indicative of the IgG antibodies exerting VEGF-D neutralizing activity. A summary of the antibodies' VEGF-D neutralizing activity (IC₅₀) is provided in Tables 11 to 15. In contrast, IgG antibodies of clones F61, HW78, HH69, HH41, HX28, HU75, DI26, and AW61 did not inhibit ³H-thymidine incorporation in the presence of VEGF-C, and thus did not exert substantial VEGF-C neutralizing activity.

TABLE 11
Summary of Experiment 1 IC₅₀ for
Selected Humanized IgG Antibodies

| | Source 1 | Source 2 | | | |
|---|---|---|---|---|---|
| Clone | VEGF-D | VEGF-D | Average | Std Dev | CV (%) |
| F61 | 1.556 × 10⁻⁵ | 2.16 × 10⁻⁵ | 1.9 × 10⁻⁵ | 4.3 × 10⁻⁶ | 23.1 |
| HW78 | 1.386 × 10⁻⁵ | 1.18 × 10⁻⁵ | 1.3 × 10⁻⁵ | 1.5 × 10⁻⁶ | 11.4 |
| HH69 | 4.951 × 10⁻⁶ | 5.43 × 10⁻⁶ | 5.2 × 10⁻⁶ | 3.4 × 10⁻⁷ | 6.5 |
| HH41#1 | 3.322 × 10⁻⁵ | 1.40 × 10⁻⁵ | 2.4 × 10⁻⁵ | 1.4 × 10⁻⁵ | 57.7 |
| HH41#2 | 2.848 × 10⁻⁵ | 1.90 × 10⁻⁵ | 2.4 × 10⁻⁵ | 6.7 × 10⁻⁶ | 28.3 |
| HX28 | 7.267 × 10⁻⁶ | 1.53 × 10⁻⁶ | 4.4 × 10⁻⁶ | 4.1 × 10⁻⁶ | 92.3 |
| HU75 | 4.035 × 10⁻⁵ | 1.53 × 10⁻⁵ | 2.8 × 10⁻⁵ | 1.8 × 10⁻⁵ | 63.5 |
| DI26 | 1.809 × 10⁻⁵ | 5.21 × 10⁻⁶ | 1.2 × 10⁻⁵ | 9.1 × 10⁻⁶ | 78.2 |
| AW61 | 2.750 × 10⁻⁵ | 1.02 × 10⁻⁵ | 1.9 × 10⁻⁵ | 1.2 × 10⁻⁵ | 64.9 |

The top 4 IgG antibodies amongst those derived from clones F61, HW78, HH69, HH41, HX28, HU75, DI26, and AW61 selected on the basis of Experiment 1 IC₅₀ and K_{D} of Source 1 VEGF-D are summarized in Table 12.

TABLE 12
Top Candidate Humanized IgG Antibodies

IC₅₀

| | Source 1 | Source 2 | | |
|---|---|---|---|---|
| Top 4 | VEGF-D | VEGF-D | Average | Dimer K_{D} |
| 1 | HH69 | HX28 | HH69 | HX28 |
| 2 | HX28 | DI26 | HX28 | DI26 |
| 3 | HW78 | HH69 | DI26 | HH69 |
| 4 | F61 | AW61 | HW78 | HW78 |

The Experiment 1 IC₅₀ for each of the IgG antibodies of clones F61, HW78, HH69, HH41, HX28, HU75, DI26, and AW61 was calculated and plotted against their respective K_{D} values (FIG. 37). The antibodies cluster in three groups, with the best antibodies exhibiting the lowest IC₅₀ and K_{D} values, i.e. the bottom left of FIG. 37. According to this metric, the top 4 IgG antibodies from amongst this group of eight are HW78, HH69, HX28, and DI26.

TABLE 13
Experiments 1 and 2 IC₅₀ and Ranking for Selected
Humanized IgG Antibodies According to VEGF-D Source

| | Source 1 | | | | Source 2 | | | |
|---|---|---|---|---|---|---|---|---|
| | Expt 1 | | Expt 2 | | Expt 1 | | Expt 2 | |
| Clone | IC₅₀(×10⁻⁵) | Rank | IC₅₀(×10⁻⁵) | Rank | IC₅₀(×10⁻⁵) | Rank | IC₅₀(×10⁻⁵) | Rank |
| HX28 | 0.727 | 2 | 0.902 | 1 | 0.153 | 1 | 0.678 | 1 |
| HH69 | 0.495 | 1 | 1.79 | 5 | 0.543 | 3 | 1.49 | 8 |
| HW78 | 1.39 | 3 | 1.05 | 2 | 1.18 | 5 | 0.686 | 2 |
| DI26 | 1.81 | 5 | 1.08 | 3 | 0.521 | 2 | 1.06 | 4 |
| HH41 | 3.08 | 7 | 3.15 | 8 | 1.65 | 7 | 0.867 | 3 |
| F61 | 1.56 | 4 | 2.32 | 7 | 2.16 | 8 | 1.35 | 7 |
| HU75 | 4.04 | 8 | 2.16 | 6 | 1.53 | 6 | 1.19 | 5 |
| AW61 | 2.75 | 6 | 1.19 | 4 | 1.02 | 4 | 1.21 | 6 |

TABLE 14
Experiments 1 and 2 Average IC₅₀ and Ranking for Selected Humanized
IgG Antibodies According to VEGF-D Source and According to Experiment

| | Source 1 | | Source 2 | | Expt 1 | | Expt 2 | |
|---|---|---|---|---|---|---|---|---|
| Clone | IC₅₀(×10⁻⁵) | Rank | IC₅₀(×10⁻⁵) | Rank | IC₅₀(×10⁻⁵) | Rank | IC₅₀(×10⁻⁵) | Rank |
| HX28 | 0.814 | 1 | 0.416 | 1 | 0.440 | 1 | 0.790 | 2 |
| HH69 | 1.14 | 2 | 1.02 | 4 | 0.520 | 2 | 0.163 | 1 |
| HW78 | 1.22 | 3 | 0.933 | 3 | 1.30 | 4 | 0.860 | 3 |
| DI26 | 1.44 | 4 | 0.791 | 2 | 1.20 | 3 | 1.07 | 4 |
| HH41 | 3.11 | 8 | 1.26 | 6 | 2.40 | 6 | 2.01 | 8 |
| F61 | 1.94 | 5 | 1.75 | 8 | 1.90 | 5 | 1.83 | 7 |
| HU75 | 3.10 | 7 | 1.36 | 7 | 2.80 | 7 | 1.67 | 6 |
| AW61 | 1.97 | 6 | 1.11 | 5 | 1.90 | 5 | 1.20 | 5 |

TABLE 15
Experiments 1 and 2 Overall Average IC₅₀ and
Ranking for Selected Humanized IgG Antibodies

| Clone | IC₅₀(×10⁻⁵) | Rank |
|---|---|---|
| HX28 | 0.615 | 1 |
| HH69 | 1.08 | 2 |
| HW78 | 1.08 | 2 |
| DI26 | 1.12 | 3 |
| HH41 | 2.19 | 6 |
| F61 | 1.84 | 5 |
| HU75 | 2.23 | 7 |
| AW61 | 1.54 | 4 |

### Example 7

Testing Neutralizing Activity of IgG Antibodies in VEGFR-3-Ba/F3 ³H-Thymidine Incorporation Bioassay

Background. The VEGFR-3-Ba/F3 cell line expresses the chimeric receptor consisting of the extracellular domain of VEGFR-3 and the transmembrane and cytoplasmic domains of EpoR. Ba/F3 cells are IL-3 dependent. In the absence of IL-3, the VEGFR-3-Ba/F3 cells survive and proliferate only in the presence of growth factors capable of binding and cross-linking the extracellular domain of mouse VEGFR-3. Thus, this assay is used to assess the ability of the antibodies described herein to neutralize VEGFR-3 activity.

Ba/F3 cells are stably transfected with a chimeric receptor containing the extracellular domain of human VEGFR-3 and the transmembrane and cytoplasmic domains of the mouse erythropoietin receptor (EpoR) (Achen et al. 2000 Eur J Biochem 267: 2505-2515). The chimeric receptor is made by introducing a BglII restriction enzyme site at the junction of the regions encoding the extracellular and transmembrane domains of the mouse EpoR cDNA using site-directed mutagenesis. Prior to this, a silent mutation is introduced into the EpoR cDNA in a region encoding the cytoplasmic domain of the EpoR to eliminate a naturally occurring BglII site. The fragment of the EpoR cDNA encoding the transmembrane and cytoplasmic domains, subcloned in plasmid pcDNA1/Amp (Invitrogen), is then ligated at the BglII site with a PCR product, consisting of DNA encoding the entire extracellular domain of human VEGFR-3, to generate a cDNA encoding a fusion protein consisting of the VEGFR-3 extracellular domain and the transmembrane and cytoplasmic domains of EpoR. The DNA fragment encoding the extracellular domain of human VEGFR-3 can be amplified by PCR using primers 5′-TAGAAAGCTTAATCTAGAGCCACCATGCAGCGGGGCG (SEQ ID NO: 32) and 5′-TAGAGGATCCCTCCATGCTGCCCT (SEQ ID NO: 33) and ligated as a HindIII-BamHI fragment into HindIII-BglII sites of the EpoR plasmid construct. The DNA encoding the chimeric receptor is subcloned into the expression vector pEF-BOS and cotransfected into the Ba/F3 cell line with pgk-Neo (a plasmid containing a neomycin resistance gene under the control of the promoter of the mouse phosphoglycerate kinase-1 gene), at a ratio of 20:1. Transfected cells are selected in G418, and a cell line expressing the VEGFR-3-EpoR chimeric receptor (Mᵣ≈450,000) is identified by immunoprecipitation and western blot analysis with antihuman VEGFR-3 polyclonal antibody (R & D Systems, Minneapolis, Minn., USA). Expression of the chimeric receptor is confirmed by flow cytometry using mAb 9D9 specific for the extracellular domain of human VEGFR-3. The cell line expressing the receptor is designated Ba/F3-VEGFR-3-EpoR.

The chimeric molecule is used because members of the receptor-type tyrosine kinase family signal poorly in hematopoietic cells such as Ba/F3, whereas signaling from the EpoR cytoplasmic domain leads to cell survival and proliferation in the absence of IL-3. Ba/F3-VEGFR-3-EpoR can be rescued with a known VEGFR-3 ligand, for example VEGF-D or VEGF-C, in the absence of IL-3. In contrast, the parental cell line, which does not express VEGFR-3, does not respond to the known VEGFR-3 ligand, indicating that the response of the Ba/F3-VEGFR-3-EpoR cell line to the known ligand is dependent on the chimeric receptor.

Candidate antibody compositions are screened for the capacity to inhibit the activation of VEGFR-3 in such a cell proliferation bioassay using Ba/F3-VEGFR-3-EpoR cells. Compounds that reduce binding of the known ligand (e.g., mature VEGF-D) to the extracellular domains of the chimeric receptors or the subsequent cross-linking of the extracellular domains will cause cell death in the absence of IL-3, even in the presence of the known ligand.

Samples of a known ligand for VEGFR-3 are incubated with a candidate composition for 1 h at 4° C. in NaCl/Pᵢ before dilution of the mixtures 1:10 with cell culture medium (e.g., Dulbecco's modified Eagle's medium containing 10% (v/v) fetal bovine serum, 50 mM L-glutamine, 50 μgmL⁻¹ gentamicin, 1 mgmL⁻¹ G418) deficient in IL-3. The resulting media will contain approximately 500 ngmL⁻¹ of known ligand for VEGFR-3 and varying concentrations of the candidate compound. Ba/F3-VEGFR-3-EpoR cells are then incubated in the media for 48 h at 37° C. DNA synthesis is quantified by addition of 1 μCi of ³H-thymidine and further incubation for 4 h prior to harvesting, for example, using an automated cell harvester. Incorporated ³H-thymidine is measured by β scintillation counting.

### Example 8

Testing Neutralizing Activity of IgG Antibodies in VEGFR-2-Ba/F3 and VEGFR-3-Ba/F3 ³H-Thymidine Incorporation Bioassays

Methodology. Eight IgG antibodies derived from clones F61, HW78, HH69, HH41, HX28, HU75, DI26, AW61 were tested according to Examples 6 and 7 for their ability to block receptor activation by three forms of human VEGF-D (Sources 1, 2 and 3) in the VEGFR-2-Ba/F3 (Example 6) and VEGFR-3-Ba/F3 (Example 7) bioassays. A neutralizing monoclonal antibody and a soluble VEGFR-3 receptor were used as positive controls (not shown).

A fixed amount of the VEGF-D proteins was mixed with different amounts of the antibodies, yielding concentrations of 500 ng/ml for Source 1 and Source 2 VEGF-D, 1250 ng/ml for Source 3 VEGF-D, and 0-36,000 ng/ml for each antibody. Fifty microliters was plated in triplicate in 96-well microtitre plates. The VEGF-D concentrations needed to achieve a measurable proliferation/survival response was determined experimentally for VEGFR-2-Ba/F3 cells and adjusted taking into account the availability of the protein. An equal volume of VEGFR-2-Ba/F3 or VEGFR-3-Ba/F3 cell suspension (400,000 cells/ml) was added (resulting in a total culture volume of 100 μl/well). After two days, 10 μl of a 5 mg/ml MTT in PBS solution were added to each well and after another 2 hours of incubation cells were lysed by adding 100 μl 10% SDS/10 mM HCl solution. After incubation overnight at 37° C. and 2 hours of agitation, the OD (540 nm) was measured with a microtitre plate spectrophotometer.

Results. All eight antibodies inhibited activation of both VEGFR-2 and VEGFR-3 by all three of the tested VEGF-D proteins. Both Source 1 and Source 2 VEGF-D were inhibited almost to baseline at high antibody concentrations in the VEGFR-2 bioassay. Inhibition of the Source 1 and Source 2 VEGF-D worked best in the VEGFR-3 bioassay. The leading antibody in the VEGFR-3 bioassay was HX28, which came close to the control VEGFR-3 soluble receptor in inhibiting Source 1 and Source 2 VEGF-D. HX28 and DI26 were the best antibodies in inhibiting Source 3 VEGF-D in the VEGFR-3 bioassay. The qualitative results of the VEGFR-3-Ba/F3 bioassay of Example 8 are presented in Table 16. The top three ranked antibodies are the same as those in Tables 12 and 15 of Example 6.

TABLE 16
Humanized IgG Antibodies Ranked in VEGFR-3-Ba/F3 bioassay

| | Source 1 | Source 2 |
|---|---|---|
| Clone | VEGF-D | VEGF-D |
| HX28 | 1 | 1 |
| DI26 | 2 | 2 |
| HH69 | 3 | 3 |
| AW61 | 6 | 4 |
| HW78 | 4 | 7 |
| HU75 | 7 | 5 |
| F61 | 5 | 8 |
| HH41 | 8 | 6 |

### Example 9

Commercial Scale Antibody Production

The IgG antibodies derived from clones HH69, HX28 and DI26 were produced on a commercial scale.

1. An isolated antibody that specifically binds vascular endothelial growth factor-D (VEGF-D) comprising a heavy chain variable (V_{H}) domain amino acid sequence provided as SEQ ID NO: 1 or a variant thereof which contains from 1 to 10 amino acid substitutions.

2-3. (canceled)

4. The antibody of claim 1, wherein the V_{H} domain amino acid sequence comprises a variant of SEQ ID NO: 1, wherein the one to ten amino acid substitutions:
(i) occur within SEQ ID NO: 1 at one or more positions selected from the group consisting of :
(a) 1, 2, 3, 5, 9, 10, 11, 12, 13, 16, 17, 18, 19, 20, 23, 24, 25, 28, 40, 43, 46, 48, 55, 63, 65, 67, 68, 69, 70, 71, 72, 73, 74, 75, 76, 77, 81, 82, 83, 84, 87, 88, 89, 93, 95, 99, 100, 110, 112, 113, 114 or 117;
(b) 2, 3, 5, 10, 12, 17, 18, 19, 20, 23, 25, 40, 46, 48, 55, 63, 65, 68, 69, 73, 75, 77, 81, 82, 89, 95, 99, 100, 112, 114 or 117; and
(c) 10, 20, 25, 55, 77 or 82; or
(ii) are selected from the group consisting of:
(d) Q1E; V2A; Q3R; V5A; A9S; E10K; V11L; K12R; Q13K; A16E; S17N; S17G; V18M; V18L; K19R; K19T; V20A; V20I; K23E; A24G; S25G; S25R; T28S; A40M; A40V; Q43K; E46G; M48V; N55S; K63R; K65R; R67Q; V68A; V68F; T69A; T69V; M70F; M70I; T71S; T72A; T72L; D73G; T74K; S75N; T76V; T76I; S77G; M81V; M81L; E82K; E82Q; L83I; L83W; R84S; R87K; S88A; D89G; D89E; D89S; V93M; Y95H; T99A; S100G; R110Q; T112A; L113T; V114A; or A117S
(e) V2A; Q3R; V5A; E10K; K12R; S17N; S17G; V18M; K19R; K19T; V20A; K23E; S25G; S25R; A40V; E46G; M48V; N55S; K63R; K65R; V68A; D73G; S75N; S77G; M81V; E82K; D89G; Y95H; T99A; S100G; T112A; V114A; or A117S
(f) E10K; V20A; S25R; N55S; V68A; S77G; and E82K;
(g) E10K, V20A and N55S; and
(h) E55S.

5-12. (canceled)

13. The antibody of claim 1, wherein the V_{H} domain amino acid sequence comprises a variant of SEQ ID NO: 1 that comprises the amino acid substitutions E10K, V20A and N55S.

14. The antibody of claim 1, wherein the V_{H} domain amino acid sequence comprises the SEQ ID NO: 1 variant that comprises the sequence provided as SEQ ID NO: 13.

15. (canceled)

16. The antibody of claim 1, wherein the V_{H} domain amino acid sequence comprises the SEQ ID NO: 1 variant that comprises the sequence provided as SEQ ID NO: 7, SEQ ID NO: 9, SEQ ID NO: 11 or SEQ ID NO: 15.

17. The antibody of claim 1, comprising a light chain variable (V_{L}) domain amino acid sequence provided as SEQ ID NO: 5 or a variant thereof which contains from 1 to 10 amino acid substitutions.

18-19. (canceled)

20. The antibody of claim 17, wherein the V_{L} domain amino acid sequence comprises a variant of SEQ ID NO: 5 that contains 1 or 2 amino acid substitutions.

21. The antibody of claim 18, wherein the V_{L} domain amino acid sequence comprises a variant of SEQ ID NO: 5 that comprises an amino acid substitution at position 5, 11, 13, 25, 32, 58, 60, 65, 75, 79, 88, 97, 105 or 108.

22. The antibody of claim 17, wherein the V_{L} domain amino acid sequence comprises a variant of SEQ ID NO: 5, wherein the one to ten amino acid substitutions occur within SEQ ID NO: 5 at one or more positions selected from the group consisting of 5, 11, 13, 25, 32, 58, 60, 65, 75, 79, 88, 97, 105 and 108.

23-24. (canceled)

25. The antibody of claim 17, wherein the V_{L} domain amino acid sequence comprises a variant of SEQ ID NO: 5 that comprises the sequence provided as SEQ ID NO: 17, SEQ ID NO: 19, or SEQ ID NO: 21.

26. The antibody of claim 17, wherein the V_{H} and V_{L} domains include:
(a) SEQ ID NO: 1 and SEQ ID NO: 5;
(b) SEQ ID NO: 1 variant E82K (SEQ ID NO: 7) and SEQ ID NO: 5;
(c) SEQ ID NO: 1 variant E10K and V20A (SEQ ID NO: 9) and SEQ ID NO: 5;
(d) SEQ ID NO: 1 variant V68A and E82K (SEQ ID NO: 11) and SEQ ID NO: 5;
(e) SEQ ID NO: 1 variant E10K, V20A and N55S (SEQ ID NO: 13) and SEQ ID NO: 5;
(f) SEQ ID NO: 1 variant S25R, S77G and E82K (SEQ ID NO: 15) and SEQ ID NO: 5;
(g) SEQ ID NO: 1 variant E82K (SEQ ID NO: 7) and SEQ ID NO: 5 variant S11G and Q105R (SEQ ID NO: 17);
(h) SEQ ID NO: 1 variant E82K (SEQ ID NO: 7) and SEQ ID NO: 5 variant S32G (SEQ ID NO: 19);
(i) SEQ ID NO: 1 variant E10K, V20A and N55S (SEQ ID NO: 13) and SEQ ID NO: 5 variant S11G and Q105R (SEQ ID NO: 17); or
(j) SEQ ID NO: 1 variant E10K, V20A and N55S (SEQ ID NO: 13) and SEQ ID NO: 5 variant D65G (SEQ ID NO: 21).

27-36. (canceled)

37. An isolated nucleic acid molecule that comprises a nucleotide sequence encoding the antibody of claim 1.

38. (canceled)

39. An isolated host cell comprising a nucleic acid molecule that comprises a nucleotide sequence encoding the antibody of claim 1.

40-44. (canceled)

45. A pharmaceutical composition comprising the antibody of claim 1 or a nucleic acid molecule of claim 37, and a pharmaceutically acceptable carrier.

46-53. (canceled)

54. The antibody of claim 1, wherein the V_{H} domain amino acid sequence comprises a variant of SEQ ID NO: 1, wherein the one to ten amino acid substitutions are selected from the group consisting of:
(a) Q1E; V2A; Q3R; V5A; A9S; E10K; V11L; K12R; Q13K; A16E; S17N; S17G; V18M; V18L; K19R; K19T; V20A; V20I; K23E; A24G; S25G; S25R; T28S; A40M; A40V; Q43K; E46G; M48V; N55S; K63R; K65R; R67Q; V68A; V68F; T69A; T69V; M70F; M70I; T71S; T72A; T72L; D73G; T74K; S75N; T76V; T76I; S77G; M81V; M81L; E82K; E82Q; L83I; L83W; R84S; R87K; S88A; D89G; D89E; D89S; V93M; Y95H; T99A; S100G; R110Q; T112A; L113T; V114A; and A117S;
(b) V2A; Q3R; V5A; E10K; K12R; S17N; S17G; V18M; K19R; K19T; V20A; K23E; 525G; S25R; A40V; E46G; M48V; N55S; K63R; K65R; V68A; D73G; S75N; S77G; M81V; E82K; D89G; Y95H; T99A; S100G; T112A; V114A; and A117S;
(c) E10K; V20A; S25R; N55S; V68A; S77G; and E82K;
(d) E10K, V20A and N55S; and
(e) E55S.

55. The method of claim 17, wherein the V_{L} domain amino acid sequence comprises a variant of SEQ ID NO: 5, wherein the one to ten selected from the group consisting of:
(a) T5I; S11G; V13A; S25T; S32G; N585; F605; D65G; T75A; K79R; V88M; T97A; Q105R; and T108A; or
(b) S11G, S32G, D65G and Q105R.
